(19) United States
(12) Patent Application Publication
(10) Pub. No.: US 2013/0177623 A1
(43) Pub. Date: Jul. 11, 2013

(54) Preparation Rich in Growth Factor-Based Fibrous Matrices for Tissue Engeering, Growth Factor Delivery, and Wound Healling

(71) Applicants: Gary L. Bowlin, Mechanicsville, VA (US); Patricia S. Wolfe, Fort Worth, TX (US); Scott A. Sell, St. Louis, MO (US)

(72) Inventors: Gary L. Bowlin, Mechanicsville, VA (US); Patricia S. Wolfe, Fort Worth, TX (US); Scott A. Sell, St. Louis, MO (US)

(21) Appl. No.: 13/823,791

(22) PCT Filed: Sep. 21, 2011
(86) PCT No.: PCT/US11/52523

§ 371 Date: Mar. 15, 2013

Related U.S. Application Data
(60) Provisional application No. 61/385,336, filed on Sep. 22, 2010.

(51) Int. Cl. A61K 9/70

(52) U.S. Cl. CPC A61K 9/70
USPC 424/443; 424/530; 435/398; 264/465

(57) ABSTRACT

Activated platelet-rich plasma (aPRP) is electrospun into fibrous matrices which are used to deliver components of aPRP to a site of action in a sustained manner. The electrospun matrices are used, for example, for tissue engineering applications and for the treatment of wounds.

### BACKGROUND OF THE INVENTION

1. Field of the Invention

The invention generally relates to electrospun fibers formed from activated platelet-rich plasma (aPRP). Fiber matrices are used for sustained delivery of growth factors and other components of aPRP, for example, in applications such as tissue engineering and the treatment of wounds.

2. Background of the Invention

Platelet-rich plasma (PRP) therapy is a method of collecting and concentrating autologous platelets, through centrifugation and isolation, for the purpose of activating and releasing their growth factor-rich α- and dense granules. The discharge of these concentrated granules releases a number of growth factors and cytokines in physiologically relevant ratios, albeit in concentrations several times higher than that of normal blood, that are critical to tissue regeneration and cellular recruitment. Some of the more highly concentrated factors found within PRP include platelet derived growth factor (PDGF), transforming growth factor-β (TGF-β), vascular endothelial growth factor (VEGF), fibroblast growth factor (FGF), and epidermal growth factor (EGF). In addition, PRP has also been shown to contain a number of macrophage and monocyte mediators such as RANTES (Regulated upon Activation, Normal T-cell Expressed, and Secreted), lipoxin, and an array of interleukins.

Clinically, PRP therapy has been used to stimulate tissue growth and regeneration in a number of different tissues; effectively accelerating the healing response in patients suffering from osteochondral defects, tendon/ligament injuries, and chronic skin wounds (diabetic and pressure ulcers). Typically, these procedures involve a blood draw and centrifugation to concentrate the platelet portion, followed by a platelet activation step and the delivery of the activated PRP to the site of injury. There have been several methods reported in the literature on successfully activating and delivering PRP to an injury site, with most involving the creation of a platelet gel using thrombin or CaCl₂ (Foster et al., Am J Sports Med 2009; 37(11):2259-72; Sanchez et al., Sports Med 2009; 39(5):345-54; Alsousou et al., J Bone Joint Surg Br 2009; 91(8):987-96; Anitua et al., Trends Pharmacol Sci 2008; 29(1):37-41). These PRP gels can be easily applied to wound sites through injection or topical application.

However, studies have shown that the use of thrombin as a clotting agent can result in a rapid activation of platelets and a bolus release of growth factors, with 70% of growth factors released within 10 minutes of clotting, and nearly 100% released within 1 hour (Foster et al., 2009). This “dumping” method fails to maximize the cell stimulating potential of the PRP-derived growth factors as most are cleared before they can take effect (Lu et al., J Biomed Mater Res A 2008; 86(4):1128-36). Growth factor release from PRP gels can be slowed when the gel is formed with CaCl₂ rather than thrombin. The addition of CaCl₂ to PRP results in the formation of autogenous thrombin from prothrombin and the eventual formation of a loose fibrin matrix that will secrete growth factors over 7 days (Foster et al., 2009).

Other techniques have been evaluated for further sustaining the release of growth factors from PRP and include the use of gelatin gel microspheres (Bir et al., J Vasc Surg 2009; 50(4):870-879 e2), freeze-dried PRP (Pietramaggiori et al., Wound Repair Regen 2006; 14(5):573-80; Pietramaggiori et al., Wound Repair Regen 2007; 15(2):213-20; Pietramaggiori et al., Wound Repair Regen 2008; 16(2):218-25; Sum et al., Transfusion 2007; 47(4):672-9), and alginate beads (Lu et al., 2008). The injection of PRP gelatin gel microspheres in a mouse ischemic hind limb model demonstrated sustained release of the PRP potent angiogenic components as illustrated by an increase in perfusion, capillary density, and mature blood vessel density (Bir et al., 2006). Alginate beads were shown to be successful in delivering (based on cell proliferation) PRP-derived growth factors and cytokines over the course of 14 days (Lu et al., 2008). The use of a freeze-dried PRP powder in a dermal wound has been shown to significantly increase cellular proliferation (up to 21 days), tissue regeneration and angiogenesis in a mouse dermal wound (Pietramaggiori et al., 2006; Pietramaggiori et al., 2007; Pietramaggiori et al., 2008; Sum et al. 2007). Collectively, these studies demonstrate the importance of keeping PRP-derived growth factors and cytokines in the wound site and slowly releasing them as the wound site becomes infiltrated with reparative cells.

While a number of individual growth factors and/or cytokines have been used previously in an array of sustained release tissue engineering and regenerative medicine applications with positive results, the use of either single or multiple isolated growth factors/cytokines is often prohibitively expensive, and it can be difficult to replicate physiologically relevant quantities (Foster et al., 2009).

There is an ongoing need to provide efficacious yet cost-effective devices and methods for the sustained delivery of activated platelet-rich plasma, or components thereof, to patients who could benefit from receiving such therapy.

### SUMMARY OF THE INVENTION

The invention provides efficacious and cost-effective electrospun constructs for long term, sustained delivery of growth factors and other components of aPRP, for therapeutic purposes. The constructs comprise fibers electrospun from a solution comprising aPRP, or from a solution of aPRP plus one or more other natural or synthetic polymers (and, optionally, other additives). The fibrous constructs of the invention are advantageously highly permeable to and/or effective in recruiting infiltrating cells, making them highly desirable for use in treating various conditions (e.g. wounds) and for tissue engineering applications. Electrospun aPRP fibers differ from fibers that are merely coated with aPRP in that they are made of aPRP and therefore have different and more favorable rates of degradation and growth factor/cytokine release kinetics, compared to fibers that are simply coated with aPRP. The fibers of the invention also differ in that the base material used for electrospinning (aPRP or, in some embodiments, PRP), in contrast to the base material used to form most conventional electrospun fibers, most resembles a blood product, rather than any sort of extracellular matrix component. Additionally, the electrospinning process allows fine control of fiber size and orientation, producing or capable of producing a range of fibers similar to a native or natural extracellular matrix.

### BRIEF DESCRIPTION OF THE DRAWINGS

FIG. 1A-C. A, SEM micrographs of electrospun silk fibroin (SF), poly(glycolic acid) (PGA), and polycaprolactone (PCL) scaffolds with and without PRGF. All images were taken at 3,000×, scale bar is 10 μm. B, Graph of mean fiber diameters for SF, PGA, and PCL scaffolds with and without PRGF. C, Graph of mean pore areas for SF, PGA, and PCL scaffolds with and without PRGF. * indicates significant differences within polymer group, p<0.05.

FIG. 2A-C. Mechanical results of peak stress (A), modulus (B) and strain at break (C) for PCL and PRGF incorporated scaffolds over 28 days. For peak stress, # indicates statistically significant differences, p<0.05, between PCL:PRGF(100) scaffolds and all other scaffolds at all other time points, except for PCL:PRGF at day 1. For modulus, ̂ indicates statistically significant differences (p<0.05) between PCL (day 1) and all other PRGF incorporated scaffolds at day 1. & indicates statistical significance (p<0.05) between PCL scaffolds at day 7 and all other scaffolds at day 7 time point, except PCL:PRGF. * indicates the modulus of PCL:PRGF(10) scaffolds at day 21 is statistically different, p<0.05, from that of PCL:PRGF(10) scaffolds at day 1 and day 7. # denotes statistically significant differences, p<0.05, between PCL:PRGF(100) scaffolds and all other scaffolds at all other time points. For strain at break, % denotes statistical significance (p<0.05) between scaffolds of PCL:PRGF(10) at days 7, 14 and 28 over all other scaffolds at all other time points, except PCL:PRGF(10) scaffolds at day 1. # indicates statistically significant differences (p<0.05) between PCL:PRGF(100) scaffolds and PCL:PRGF(10) scaffolds at all time points. * indicates statistical significance (p<0.05) between PCL:PRGF(100) scaffolds at day 1 versus day 28.

FIG. 3. Results of macrophage chemotaxis to PRGF incorporated scaffolds and tissue culture plastic (TCPS) over 72 hours. * indicates statistical significance (p<0.05).

FIG. 4. Macrophage IL-10 release when cultured on PRGF incorporated scaffolds, TCPS and media supplemented with 1 mg/ml PRGF (TCPS:PRGF). IL-10 is a chemokine released by M2, or pro-regenerative, macrophages. Results are normalized to amount of IL-10 released (ng/ml) per 10,000 cells. # and * indicates statistically significant differences, p<0.05, of IL-10 released from different material groups at day 14.

FIG. 5. Macrophage TNF-α release when cultured on PRGF incorporated scaffolds, tissue culture plastic (TCPS) and media supplemented with 1 mg/ml PRGF (TCPS:PRGF). TNF-α is a chemokine released by M1, or pro-inflammatory, macrophages. Results are normalized to amount of TNF-α released (ng/ml) per 10,000 cells. * indicates statistically significant differences, p<0.05, between all other scaffolds at all other time points.

FIGS. 6A and B. A, SEM micrographs of electrospun PRGF scaffolds taken at 500×, scale bar represents 50 μm. B, SEM micrographs of electrospun PRGF scaffolds taken at 3000×, scale bar represents 5 μm.

FIG. 7. Graph of mean fiber diameters for electrospun PRGF scaffolds of different concentrations illustrating the linear relationship between PRGF concentration and fiber diameter. * denotes statistical significance.

FIG. 8. Quantification of generic protein released from pure PRGF scaffolds over 35 days. * indicates significant differences, p<0.05, for days 1 and 28 when compared to all other time points for each material, but not each other. # indicate statistically significant differences, p<0.05, between days 1, 4, and 35 when compared to other time points, but not each other. Minimum level of detection was 17 μg/ml.

FIGS. 9A and B. A, FBG fluorescence intensity of 100, 150, and 200 mg/ml pure PRGF scaffolds, 10, 5, and 1 mg/ml PRGF in PBS and blood, aPRP and PPP taken at 800 nm, 3.5 intensity. B, Quantified FBG expression on scaffolds, PRGF in water, blood, aPRP and PPP.

FIG. 10. DAPI staining of ADSCs cultured on electrospun scaffolds of pure PRGF at days 3 and 10. Images at 20×.

FIG. 11. DAPI staining of SMCs cultured on electrospun scaffolds of pure PRGF at days 3 and 10. Images at 20×.

FIGS. 12A and B. 80 mg/mL silk:250 mg/mL PRGF:50 mg/ml PEO surface at 1 kx (A) and 3 kx (B).

FIG. 13A-D. 80 mg/mL silk:300 mg/mL PRGF:75 mg/ml PEO. A, inside surface at 1000×; B, outside surface at 1000×; C, inside surface at 3000×; D, outside surface at 3000×.

FIGS. 14A and B. Silk:PRGF (300 mg/mL) crosslinked with EDC (A) and genipin (B)

FIG. 15A-D. 300 mg/mL PRGF:75 mg/ml PEO. A, inside surface at 1000×; B, outside surface at 1000×; C, inside surface at 3000×; D, outside surface at 3000×.

FIG. 16A-C. Comparison of the properties of crosslinked silk, PRGF and blended scaffolds. A, the average modulus; B, the average strain at break; C, the average peak stress.

### DETAILED DESCRIPTION

According to the invention, electrospun fibers comprising (i.e. formed from) a solution of aPRP, or from a solution of aPRP plus one or more additional polymers, are manufactured and used therapeutically to treat various conditions and/or for tissue engineering purposes. Release of the aPRP components from scaffolds formed from the fibers is advantageously slow, i.e. the invention provides long-term, sustained release of the components from a single fibrous matrix over a period of at least days, usually weeks, and even for a month or more, as described in detail below. As such, these fibers are distinct from fibers formed from other materials (e.g. other polymers) which merely have PRP or aPRP coated onto the fibers or attached to the fibers (e.g. by sprinkling dried PRP or aPRP onto the fibers). The fibers of the present invention are made from the PRP or aPRP. Fibers made from electrospun aPRP differ from fibers which are merely coated with aPRP not only in composition, but the differences in composition results in differences in, for example, the release kinetics of the aPRP derived growth factors/cytokines. A fiber made entirely of aPRP has a more sustained release versus a fiber coated with aPRP. Additionally, bioactivity and cell binding sites will be different; once a fiber coated with aPRP elutes its growth factors/cytokines the level of bioactivity is likely to significantly decrease, while a fiber made entirely of aPRP will maintain high levels of bioactivity throughout its duration.

By “aPRP”, we mean “activated platelet-rich plasma (PRP)”, i.e. a preparation of blood plasma that has been concentrated to increase platelet concentrations, and in which the platelets have been activated and/or lysed to release their contents.

Those of skill in the art will recognize that several methods are known for obtaining PRP. Generally, such methods involve the collection of whole blood and then suitable preparation of the blood. In some embodiments of the invention, the blood that is used is from the person who will receive or be treated with the electrospun matrix, i.e. the matrix is an autologous matrix. However, in other embodiments, the blood that is used is pooled allogenic blood and the matrices that are formed are thus allogenic, and may be used by any patient in need thereof.

Once harvested, coagulation of the blood is prevented (e.g. by the addition of sodium citrate dextrose, EDTA, oxalate, heparin, etc., and separation of platelets from platelet poor plasma and red blood cells is carried out, via, for example, one or more steps or stages of centrifugation. In humans, a typical baseline blood platelet count is approximately 200,000 per μL and the preparation of therapeutic PRP concentrates the platelets by roughly five-fold e.g. to about 1×10⁶ platelets per μL, although this range may vary according to blood source, the preparation technique and efficiency, etc. Generally, for the purposes of the invention, the concentrated PRP contains from about 1×10⁵ to about 1×10⁷ platelets per μL, or from about 5×10⁵ to about 5×10⁶ platelets per μL, or even 6, 7, 8, or 9×10⁶ or about 1, 2, 3, 4, or 5×10⁶ platelets per μL. Those of skill in the art will recognize that the concentration of platelets may vary widely, depending e.g. on the sample source and the concentration technique. Typically, a concentration in the range of about 2-10 fold, or usually about a 5-7 fold concentration is effected.

Activation of the PRP to produce activated aPRP may be accomplished by any of several methods that are known in the art, including but not limited to: by the addition of factors that naturally cause activation in vivo, e.g. by the addition of thrombin and calcium chloride; or contact with exposed collagen, adenosine triphosphate (ADP), thromboxane A2, serotonin, platelet activating factor, cytokines such as platelet factor 4 (PF4), von Willebrand factor, etc; or by physical manipulations such as freeze-thawing (as described herein); by shear stress, contact with glass beads, etc. For the practice of the present invention, a freeze-thaw cycle of freezing, thawing and then refreezing, is typically used to cause activation, e.g. freezing at −70° C. for 24 hours, thawing at 37° C. (e.g. for one hour), and then refreezing at −70° C., in order to lyse and hence activate the platelets.

In order to prepare the aPRP for electrospinning, the refrozen aPRP mixture is then dehydrated. This is generally accomplished by freeze drying, although other suitable methods may also be used, e.g. dessication carried out at higher temperatures and with or without the use of a vacuum, etc. For use in the practice of the present invention, the resulting dried mixture may then be ground, pulverized, crushed or otherwise finely dispersed into solid particles to form a fine dry powder (e.g. with a mortar and pestle, or by industrial grinding, cryogrinding, using a rotary tumbler, etc.).

aPRP contains several different growth factors and other blood proteins (e.g. other cytokines) that stimulate healing of bone, soft tissue, etc. The factors are present in high amounts due to having been concentrated through centrifugation. However, the amounts are present in physiologically relevant ratios. The components of aPRP include but are not limited to: platelet-derived growth factor (PDGF); transforming growth factor-β (TGF-β); vascular endothelial growth factor (VEGF); fibroblast growth factor (FGF), epidermal growth factor (EGF); insulin-like growth factor 1 (IGF-1); insulin-like growth factor 2 (IGF-2); keratinocyte growth factor; connective tissue growth factor; chemotactic proteins, sphingosine 1-phosphate (SIP), various macrophage and monocyte mediators such as RANTES (Regulated upon Activation, Normal T-cell Expressed, and Secreted), tumor necrosis factor α (TNFα), interferon gamma (IFNγ), and granulocyte-macrophage colony stimulating factor (GM-CSF); lipoxin; various interleukins capable of mediating inflammation (IL-8, IL-1β, IL-6, IL-10, IL-13, IL-4); proteins such as albumin and fibrinogen; various immunoglobulins; etc. The invention provides methods of releasing one or more of these components from electrospun fibers formed from aPRP, usually in a sustained (long-teem) manner.

In one embodiment, electrospun fibers are created from non-activated PRP. In this embodiment, a dehydration process of gradually lyophilizing platelets without lysing them is employed. Fibers created in this manner behave differently from fibers made from activated PRP. For example, electrospun fibers of non-activated PRP would produce a sustained release of the circulating blood proteins contained within PRP (e.g. fibrinogen, albumin, immunoglobulins, etc.), and some clinical benefit may be obtained from a sustained release of these blood proteins. Alternatively, an electrospun scaffold made from a non-activated PRP might undergo a further activation step prior to use, to render it capable of releasing growth factors/cytokines.

In order to electrospin the aPRP or PRP, the dry powder is typically dissolved in a solvent, examples of which include but are not limited to: 1,1,1,3,3,3 hexafluoro-2-proponol (HFP), water, trifluoroethanol (TFE), ethanol, saline, etc. Typically, the aPRP powder is present in the solution at a concentration of from about 1 to about 1000 mg/ml of electrospinning solution; or from about 5 to about 500 mg/ml of electrospinning solution; or from about 80-280 mg/ml, or from about 10 to about 200 mg/ml of solvent, e.g. about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 or more mg/ml of electrospinning solution.

In some embodiments, the fibers of the invention are spun from a solution that includes only aPRP in a suitable solvent. The solvent evaporates during fiber formation, so that the fibers per se are formed only from aPRP. However, in other embodiments, co-polymers are also present in the fibers. In one embodiment, one or more co-polymers is added to the aPRP solution that is electrospun. The additional polymers may be either natural or synthetic, examples of which include but are not limited to: polyurethane, polyester, polyolefin, polymethylmethacrylate, polyvinyl aromatic, polyvinyl ester, polyamide, polyimide, polyether, polycarbonate, polyacrilonitrile, polyvinyl pyrrolidone, polyethylene oxide, poly (L-lactic acid, “PLA”), poly (lactide-CD-glycoside), polycaprolactone (PCL), polyphosphate ester, poly (glycolic acid), poly (DL-lactic acid), and some copolymers (e.g. PLA co-polymers of PGA PLA, polyesters, and native proteins such as collagens, gelatin, fibronectin, fibrinogens, recombinant proteins and other natural and synthetic proteins and peptide sequences); biomolecules such as DNA, silk (e.g. formed from a solution of silk fiber and hexafluoroisopropanol or water), chitosan and cellulose (e.g. in a mix with synthetic polymers); various polymer nanoclay nanocomposites; halogenated polymer solution containing a metal compounds (e.g. graphite); memory polymers including block copolymers of poly(L-lactide) and polycaprolactone and polyurethanes, and/or other biostable polyurethane copolymers, and polyurethane ureas; linear poly(ethylenimine), grafted cellulosics, poly(ethyleneoxide), and poly vinylpyrrolidone; solutions of polystyrene (PS) in a mixture of N,N-dimethyl formamide (DMF) and tetrahydrofuran (THF) poly(vinyl pyrrolidone) (PVP) composites; poly(D,L-lactide), polyglycolide, polydioxanone, poly(trimethylene carbonate), poly(4-hydroxybutyrate), poly(ester amides) (PEA), polyurethanes, and copolymers thereof; various polyesters and acrylics; various colloidal dispersions; solutions with dispersed hydroxyapatite (HA) particles; polysulfone and a vinyl lactam polymers; dextrans and sucrose; various charged nylons (e.g. nylon 66 for protein adhesion and other variants designed to adhere to RNA and DNA); nitrocellouse; dendritic poly(ethylene glycol-lactide); etc. These materials and electrospinning techniques and variants thereof (e.g. various applications of electrospun materials, various coatings, etc.) are described, for example, in issued U.S. Pat. Nos. 6,110,590; 7,887,772; 7,824,601; 7,794,219; 7,759,082; 7,615,373; 7,575,707; 7,374,774; 7,083,854; 6,787,357; 6,753,4541; and 6,592,623; and published US patent applications 20110150973; 20110148004; 20110143429; 20110140295; 20110135901; 20110130063; 20110123592; 20110092937; 20110091972; 20110079275; 20110072965; 20110064949; 20110052467; 20100310658; 20100291058; 20080159985; 20080038352; 20050192622; 20040116032; 20040009600; and 20030207638; the complete contents of each of which are hereby incorporated by reference, as are the references cited therein. The additional polymers are typically present in amounts similar to those listed above for the dissolved aPRP, and may be present in an amount of from about 50 to 500 (e.g. 50, 60, 70, 80, 90, 100, 150, 200, 250, 300, 350, 400, 450, or 500) mg per ml of solution that is to be electrospun.

In one embodiment, the co-polymer is a polyether compound such as an oligomer or polymer of ethylene oxide. Examples of this genre of molecule include but are not limited to polyethylene glycol (PEG), polyethylene oxide (PEO) and polyoxyethylene (POE), depending on its molecular weight. The three names are chemically synonymous, but historically PEG has tended to refer to oligomers and polymers with a molecular mass below 20,000 g/mol, PEO to polymers with a molecular mass above 20,000 g/mol, and POE to a polymer of any molecular mass. The co-polymers may also be e.g. PLA—poly(lactic acid), PLGA—poly(lactic-co-glycolic acid), and/or PDO—polydioxanone.

In the practice of the invention, if co-polymers are present, a single solution may be formed for electrospinning as described above, the single solution containing both dissolved aPRP and the co-polymer. Alternatively, dual electrospinning techniques may be used, e.g. a 2-input nozzle that mixes separate solutions only at the outlet tip as electrospinning occurs; or using two separate solution reservoirs (e.g. syringes) which target the same collection mandrel. In these embodiments, the amounts of aPRP and co-polymer in the solutions that are electrospun are in the ranges described above. The composition of aPRP/co-polymer fibers may be varied in order to obtain fibers with desired characteristics, e.g. in order to vary fiber strength, rate of dissolution and/or absorption by liquid, porosity, flexibility, and other properties. For example, the synthetic polymer content may be increased to increase mechanical strength and to tailor (e.g. slow or decrease, or in some embodiments, to speed up or increase) rates of degradation and growth factor/cytokine release.

In other embodiments, fibrous mats or matrices are formed which comprise at least two types of fibers: at least one fiber type is made from a solution of aPRP or aPRP plus one or more copolymers, and at least one other fiber type does not contain aPRP, or contains fibers of a different aPRP composition. Such matrices may be referred to herein as composite matrices, and are manufactured using e.g. a dual system which produces at least two separate streams of liquid which do not mix prior to deposition on a mandrel. The resulting matrix is thus comprised of at least two different types of fibers, each type of which has a different chemical composition. As is the case for aPRP/co-polymer fibers described above, matrices of this type may be designed and varied so as to have particular characteristics such tensile strength, dissolution (degradation rate, porosity, flexibility, and to tailor (e.g. slow or decrease, or in some embodiments, to speed up or increase) rates of degradation and growth factor/cytokine release, etc.)

Various methods and techniques for making electrospun fibers are known in the art, and are described, for example, in U.S. Pat. Nos. 7,134,857; 7,297,305; 7,309,498; 7,485,591; 7,592,277; 7,828,539; 7,981,353; 7,759,082; 7,615,373; 7,374,774; 6,787,357; and 6,592,623, the complete contents of each of which are hereby incorporated by reference; as well as U.S. patent application Ser. No. 12/937,322 (published as US-2001-0150973) and International patent application PCT/US2011/042114, the complete contents of each of which are also hereby incorporated by reference. (These references also describe alternative solvents and co-polymers that may be used in the practice of the invention.)

Briefly, electrospinning uses an electrical charge to draw very fine (typically on the micro- or nano-scale) fibers from a liquid. When a sufficiently high voltage is applied to a liquid droplet, the body of the liquid becomes charged, and electrostatic repulsion counteracts the surface tension and droplet is “stretched”, whereupon a stream of liquid erupts from the surface and a charged liquid jet is formed. The jet dries in flight and is finally deposited on a grounded collector or mandrel. The elongation and thinning of the fiber leads to the formation of uniform fibers with micro- or nanometer-scale diameters. A standard laboratory apparatus for electrospinning includes a spinneret (e.g. a hypodermic syringe needle) connected to a high-voltage (e.g. 5 to 50 kV) direct current power supply, a syringe pump, and a grounded collector or mandrel. A polymer solution is loaded into the syringe and extruded from the tip, typically at a constant rate by, e.g. a syringe pump. Alternatively, the droplet at the tip of the spinneret can be replenished by feeding from a header tank providing a constant feed pressure.

In general, the conditions for electrospinning are as follows: an ambient temperature of from about 60 to about 75° F.; a relative humidity of from about 30% to about 40%, and typically at least about 20%. The electrospun fibers are “dry” and should be protected from exposure to moisture to prevent premature dissolution. However, some water is associated with the fibers and fiber compositions can contain from about 7 to about 8% water. In some embodiments, the fibers are sterilized prior to use, e.g. by rinsing in ethanol or other disinfecting substance, or by using electromagnetic radiation, for example, X-rays, gamma rays, ultraviolet light, etc. In some embodiments, the moisture content of a matrix (e.g. a bandage) should be reduced to 5% or less, in order to preserve biomolecular activity during sterilization. This can be achieved by drying the matrix, e.g., under a vacuum, or by using a fabrication method that reduces moisture content from the beginning. Typically, the fibrous matrices of the invention are sterilized using X-rays in a dose of about 5 kilograys (kGray). Any method that does not destroy the fibers or the activity of substances which make up the fibers may be used to sterilize the matrices of the invention.

The electrospun fibers of the invention generally have a size in the nanometer or mm range of cross-sectional diameter, usually on the order of from about 0.75 microns to about 1.25 microns. The diameter of the electrospun fibers is important, depending upon the intended application. Smaller diameter fibers have a more rapid release of growth factors/cytokines, while larger diameter fibers would have a slower release. Additionally, cell behavior/interaction can be dependant upon the size of the fibers with which they interact. The ability to create a range of fiber sizes is the most critical aspect for tissue engineering, as it allows for the use of the electrospun aPRP scaffolds in a range of applications.

The fibers are generally deposited as a mat of fibers, which may be referred to herein as a scaffold, matrix, construct, structure, fibrous matrix, etc. The mat usually comprises several layers of elongated fibers. Fiber orientation on the target mandrel is generally regulated by spinning conditions. For example, when a slowly rotating mandrel is used, fibers typically collect in a random fashion over the surface of the target mandrel. By increasing the rate of mandrel rotation (increased rotational velocity), fibers can be induced to deposit in an aligned manner and in a circumferential pattern about the target mandrel. If a non-conductive target mandrel is suspended between two grounded poles, as in a two pole air gap electrospinning system, the fibers can be induced to collect along the surface of the mandrel in parallel with the long axis of the cylindrical mandrel. [See: Jha B S, Colello R J, Bowman J R, Sell S A, Lee K D, Bigbee J W, Bowlin G L, Chow W N, Mathern B E, and D G Simpson. Two pole air gap electrospinning: Fabrication of highly aligned, 3D scaffolds for nerve reconstruction. Acta Biomaterials 7:203-215 (2010)]; and Sell S A, McClure M J, Ayres C E, Simpson D G, and Bowlin G L. Preliminary Investigation of Airgap Electrospun Silk-Fibroin-Based Structures for Ligament Analogue Engineering. Journal of Biomaterials Science: Polymers Edition. 22:1253-1273 (2011)]. Fibers can also be induced to collect on the target mandrel if the mandrel is placed between a source of polymer and a separate ground. Under these circumstances, fibers may be induced to form as a polymer leaves the source reservoir and passes towards the ground, and fibers will collect on the mandrel if it is placed in a position between the source of polymer and the ground, in a pattern dictated by the placement or orientation of the electrospinning components and the conditions that are used during electrospinning.

The dimensions of the fibrous matrices may vary widely, depending on the design requirements, their intended use, and how they are made. Generally, matrices have dimensions similar to those of the mandrel on which they are formed. Fibrous matrices of the invention will typically be from about 0.5 cms or less to about 30 cms or more in length and/or width, but larger or smaller sizes are also contemplated. In one embodiment, e.g. for use as a vascular graft, the length is on the order of from about 1 cm or even less to about a meter or longer, as required.

The height or thickness of a matrix can likewise vary considerably. Those of skill in the art will recognize that the thickness will vary depending on the amount or number of layers of fibers that are deposited, the dimensions of the fibers, amount of porosity that is introduced, the loft, etc., and that these factors may be varied to accord with desired characteristics of the material being formed. Generally, the height will vary e.g. from 0.5 cm or less (e.g. about 0.1, 0.2, 0.3, or 0.4 cm) up to any desired thickness, e.g. from about 1 to about 30 cm, or usually less, e.g. from about 1 to about 20 cm, or from about 1 to about 10 cm, or even from about 1 to about 5 cm, e.g. matrices with a thickness of about 1, 2, 3, 4, 5, 6, 7, 8, 9, or 10 cm are usual.

The shape of a fibrous matrix of the invention may be any that is useful, e.g. cylindrical, cone-shaped, planar, etc. Mandrels often generate cylindrical fiber mats, which may be used “as is” (i.e. the shape of the mandrel dictates the shape and size, and thus a suitable mandrel is selected for forming the mat). However, a “conveyor belt” style deposition onto a flat collection surface, leading to the creation of sheets of fibers which can be of any desired dimensions. In addition, modifications may be made to the electrospun material after formation, e.g., a tubular scaffold may be cut to form a sheet, or cut to form multiple smaller tubular scaffolds, or multiple scaffolds may be joined together to form longer structures or structures with angles, or a scaffold may be trimmed to a desired size or shape, etc., or structures with other desired properties may be formed, e.g. with regions of varying porosity, thickness, width, etc.

Those of skill in the art will recognize that electrospinning is not the only way to make aPRP fibers. Such fibers may also be produced by other methods of aerosolization, by e.g. gel/coagulation, phase separation, lyophilized gels, self assembly, etc. For example, other non-electrospinning methods used for making other types of fibers (e.g. collagen) may be employed, and the resulting construct may be a fiber, or alternatively a film, gel, block, bead, or other type of geometric structure.

In some embodiments, the electrospun matrices described herein may also contain one or more other beneficial materials or substances that are desirable, e.g. to promote the healing process, to encourage cell migration into and growth within (on, through, etc.) the matrices, to stabilize the fibers, or for any other reason. In some embodiments, the compounds may be added to the spinning solutions and thus incorporated into the fibers themselves. In other embodiments, the substances may be added exogenously, i.e. by being attached to the fibers (either chemically via chemical bonds such as covalent, ionic, or hydrophobic bonds, etc.); or mechanically, e.g. by being dried onto the fibers after the fibers are soaked in or sprayed with a solution containing the substance(s); or by simply being “sprinkled” or “sifted” in a dry form (e.g. a powder) onto fibers e.g. between layers of fibers in a matrix, or within spaces between fibers; etc. Examples of materials or compounds that may be added to the fibers include but are not limited to: heparin, growth factors and cytokines, coagulants, anti-coagulants, antibiotics, various chelators to enhance sustained release, various proteins peptides and nucleic acids, lipids, anesthetics; pain medications; preservatives, vitamins, etc.

An advantageous property of the matrices described herein is that they are capable of slowly releasing the components of which they are formed (components of aPRP) into a surrounding liquid milieu, e.g. into fluids within a tissue with which they are in contact, such as within a mammalian body. In contrast to prior art electrospun constructs, the bioactive molecules which are delivered by the matrices of the invention are not merely associated with the matrix (e.g. layered onto or attached to the fibers), but the matrix is actually formed from the bioactive molecules. As a result, as the fibers themselves degrade or dissolve within (as a result of exposure to or contact with) a liquid, the biomolecules which make up the fibers are released into the liquid. The matrices of the invention are made up of fibers, or combinations of fibers, that cause the matrix to dissolve relatively slowly, thus providing long-term, sustained release of the biomolecules. By “long-term” or “sustained” release, we mean that the fibers which make up the matrices of the invention generally dissolve, when in an aqueous environment (such as within the body or when otherwise in contact with body fluids) over a period of time ranging from about 1 to about 60 days, or from about 7 to about 30 days, e.g. over a period of about 7, 8, 9, 10, 11, 12, 13, 14, 15, 16, 17, 18, 19, 20, 21, 22, 23, 24, 25, 26, 27, 28, 29 or 30 days, or even longer (e.g. about 31, 32, 33, 34, 35, 36, 37, 38, 39, or 40 days). As the fibers dissolve, the active biomolecules that make up the fibers (components of aPRP, plus any other desired bioactive substances) are released in concentrations which are higher than usual physiological concentrations, but which are at normal physiological ratios, since they are derived from a natural biological source (e.g. mammalian blood) which has been concentrated. Due to the slow rate of release from the fibers, the higher than normal concentrations of biomolecules is sustained for lengthy periods of time, e.g. from about 1 to about 60 days, or from about 7 to about 30 days, e.g. over a period of about 7, 8, 9, 10, 11, 12, 13, 14, 15, 16, 17, 18, 19, 20, 21, 22, 23, 24, 25, 26, 27, 28, 29 or 30 days, or even longer (e.g. about 31, 32, 33, 34, 35, 36, 37, 38, 39, or 40 days). By a “higher than normal (or average) concentration” we mean that the measurable levels (the local concentration) of at least one of the aPRP components is about 2-10, e.g. 2, 3, 4, 5, 6, 7, 8, 9, or 10 or even more) times greater than the level that is measured at a comparable location which has not been exposed to the fibers of the invention.

The electrospun materials described herein may be utilized for a variety of applications. In some embodiments, they are used for tissue engineering endeavors, e.g. to prepare “artificial” organs or clusters of cells which perform part or all of the function of an organ, e.g. heart, pancreas, liver, skin, skeletal muscle, cardiac muscle, intestine, bowel, esophagus, trachea and other hollow organs, nerve, bone, etc. In some embodiments, a combination of cells (which may be added exogenously to a support or may originate in a patient's body), engineering, materials, and (optionally) additional suitable biochemical and physio-chemical factors are used to improve or replace biological structures, particularly structures that are injured, damaged or missing, or that need to be removed and replaced. “Tissue engineering” covers a broad range of applications, but is generally associated with applications that repair or replace portions of or whole tissues (i.e., bone, cartilage, blood vessels, bladder, skin, etc.). Often, the tissues involved require certain mechanical and structural properties for proper preparation prior to and/or during in vivo use. “Tissue engineering” encompasses efforts to perform specific biochemical functions using cells within an artificially-created support system provided by e.g. a fibrous scaffold or matrix such as those of the invention (e.g. an artificial pancreas, liver, kidney, etc.). The term “regenerative medicine” may be used synonymously with “tissue engineering”.

With respect to tissue engineering, as an example, an acellular dermal regeneration template may be made from electrospun aPRP fibers. Such a material would chemotactically attract autologous cells and promote in situ tissue regeneration. However, there may be applications where it would be beneficial to pre-seed the scaffolds with cells to provide some in vitro regeneration prior to implantation in the body.

In some embodiments, a scaffold that is not pre-seeded with cells is implanted in or on a subject in need thereof to supply the structural properties of missing or damaged organs and/or tissues. For example, such scaffolds may be used as stents, stent coatings or vascular grafts to replace or bypass blood vessels. In this embodiment of in situ regeneration, the cells which infiltrate the support come from internal body tissue, as do the physiological factors that interact with the cells (although drugs or active agents may also be added to the support before implantation, e.g. agents which stimulate angiogenesis). Cells from the recipient's body infiltrate porous areas of the scaffold after it is implanted and, using the scaffold as support, migrate within the scaffold and undergo cell division and differentiation within or on the scaffold, eventually forming a substitute tissue/organ (or a mass of cells that functions as a substitute organ or tissue) that has at least some beneficial attributes or capabilities of the organ/tissue that has been replaced, or whose function is being augmented. In this embodiment, the fibrous matrices of the invention are used as supports for the regrowth of new tissues or cells or even organs, or serve e.g. as nerve guides, as templates or mimetics to facilitate regrowth of skin and other connective tissues like ligament and tendon, in cosmetic surgery and/or reconstructive surgery, etc. The structures provided by the invention thus have uses both in vitro and in vivo.

In other embodiments of the invention, the fibrous matrices described herein are used as or formed into bandages or dressings, usually for the treatment of wounds. The site of action (usually the wound) contains or will contain a liquid (e.g. a body fluid such as blood), and when the bandage is applied to the site of action, the fibers of the bandage eventually dissolve in the liquid, releasing the active components in a sustained manner, as described herein. In one embodiment, the site of action is a wound bed, and the fibrous matrix aids in promoting clotting of blood and subsequent healing of the wound. In some embodiments, the fibers of the bandage eventually dissolve completely so there is no need to remove the bandage from the site of action. In other embodiments, the bandage may comprise other materials such as support or backing material which may later be removed as necessary. Those of skill in the art will recognize that, for the purpose of treating wounds, the shape of a fibrous matrix may be designed in any of several desirable ways, e.g. flat to be applied to surface wounds, cylindrical to be applied to puncture wounds, or even flexible to conform to the contours of any or most wounded surfaces.

In yet other embodiments, fibers of aPRP are electrospun directly onto a dermal wound (e.g. a burn, surgical incision, chronic ulcer, etc.) of a patient to serve for example as a template for dermal regeneration or as a hemostatic agent. This might occur, for example, at a patient's bedside, in an operating theatre, at a location where an injury occurred (e.g. at the location of an accident or fire, on the battlefield, etc., prior to transfer of the patient to a medical facility). Utilizing an aqueous solution to dissolve the lyophilized PRP, a clinically relevant aPRP scaffold is created without exposing the patient to harsh organic solvents which may otherwise be used in the electrospinning process. It has been shown that fibers of aPRP can be created from a range of concentrations when electrospun from water, either by itself, or when combined with other polymers (e.g. silk fibroin, PEO, etc.). Such a process is carried out using a handheld/portable electrospinning apparatus, consisting of a reservoir for holding the dissolved aPRP solution, an outlet for dispensing the fibers, and a power supply capable of supplying the necessary voltage while the patient is grounded.

The invention also provides methods of treating various medical conditions, diseases, etc. which could benefit from the sustained, long-term release growth factors and/or other components of PRP and or aPRP. Conditions which may be treated using the fibers and methods of the invention include but are not limited to: treatment of wounds (e.g. open wounds, puncture wounds, scrapes, cuts, etc.), either non-intentional on the part of the victim (e.g. resulting from accidents, falls, gunshot wounds, wounds received in combat, sports injuries, etc.) or intentionally (e.g. surgical wounds, such as result from plastic surgery, oral surgery, or any other surgical wound; etc.); nerve injury; tendon/ligament injuries; cardiac muscle injury; bone repair and regeneration; osteochondral defects; chronic skin wounds (e.g. diabetic and pressure ulcers; hemostatic devices/bandage, cartilage repair, vascular grafting, skeletal muscle, synovium, fasciae, any application where particular attention to angiogenesis and accelerated regeneration would be necessary, etc.

The fibers and methods of the invention are generally used to treat mammals, (although this is not always the case). The mammal is sometimes, but not always, a human; veterinary applications are also encompassed by the invention. Wounds or injuries of e.g. companion pets, of other non-human animals (especially those of commercial value such as horses, various types of livestock, etc.) may also be treated by and benefit from the practice of the invention.

### EXAMPLES

### Example 1

### Reparative Potential of Platelet Rich Plasma (PRP) as Applied to Tissue Engineering Via the Creation of PRP Eluting Electrospun Scaffolds

### Introduction

The purpose of this study was to harness the reparative potential found in PRP, namely the growth factor and cytokine milieu contained within, and apply it to tissue engineering through the creation of a PRP eluting electrospun scaffold. Lysed and lyophilized PRP formed an effective “preparation rich in growth factors” (PRGF) capable of being introduced into the electrospinning process to create a scaffold with enhanced bioactivity capable of a sustained release of growth factors.

### Materials and Methods

### Creation of PRP and PRGF

Fresh human whole blood from 3 donors was purchased (Biological Specialty Corp.), pooled, and used in a SmartPReP® 2 (Harvest Technologies Corp.) centrifugation system to create PRP per manufacturers protocol. A small aliquot of both pooled whole blood and PRP were sent to the Harvard Immune Disease Institute's Blood Research laboratory to determine their respective platelet concentrations. PRP was then subjected to a freeze-thaw-freeze (FTF) cycle in a −70° C. freezer for cell lysis and activation. Centrifuge tubes containing PRP were placed in a −70° C. freezer for 24 hrs followed by a 37° C. waterbath for 1 hr, and then returned to the −70° C. freezer for 24 hrs. The degree of activation of the FTF lysed PRP, and thrombin (Recothrom, ZymoGenetics Inc.) and 10% CaCl₂ (American Regent) activated PRPs was quantified through an enzyme-linked immunosorbent assay (ELISA) analysis of VEGF and bFGF (Antigenix America Inc.). Frozen PRP was then lyophilized for 24 hrs to create a dry PRGF powder which was finely ground in a mortar and pestle prior to use.

### Chemotactic Effect of PRGF on Macrophages

To determine the role that powdered PRGF had on human macrophage chemotaxis, and to demonstrate that lyophilized PRGF retained viable chemotactic proteins, PRGF was dissolved in macrophage serum-free media (MSFM, Invitrogen) in a range of concentrations (0, 0.01, 0.1, 1, 5, and 10 mg/ml). Using a 24-well Transwell plate with 8 μm diameter pores (Corning, Inc.), 600 μl of PRGF containing media was placed in the bottom wells, while the top insert was seeded with 100,000 human peripheral blood macrophages (ATCC, CRL9855) in 150 μl of control media for 18 hours (n=3). Following the 18 hour duration, the bottom wells were aspirated and the average cell number was determined through the use an automated cell counter (Invitrogen Countess).

### Creation of PRGF Eluting Electrospun Scaffolds

Scaffolds used in this study consisted of silk fibroin (SF, extracted from bombyx morii silkworm cocoons), poly (glycolic acid) (PGA, Alkermes), and polycaprolactone (PCL, Lakeshore Biomaterials, 125 kDa). Each of these materials was dissolved in 1,1,1,3,3,3 hexafluoro-2-propanol (HFP, TCI America Inc.) at a concentration of 100 mg/ml to create the solutions used in the electrospinning process. These materials were chosen as they are three commonly used biomaterials that have been well characterized, and have well known degradation characteristics.

To each of these electrospinning solutions PRGF was added in concentrations of 10 or 100 mg of PRGF per ml of electrospinning solution (SF:PRGF(10), PGA:PRGF(10), and PCL:PRGF(10) and SF:PRGF(100), PGA:PRGF(100), and PCL:PRGF(100), respectively) and allowed to dissolve completely into solution. As PRGF can be successfully electrospun by itself from HFP at a concentration of 200 mg/ml, PRGF fibers were also integrated into PCL scaffolds using two additional electrospinning techniques: 1) a 2 input-1 output nozzle that mixed separate PCL and PRGF solutions only at the outlet tip as electrospinning occurred (PCL:PRGF(2-1)) and 2) using two separate syringes of PCL and PRGF electrospinning solutions at 90° from each other targeting the same collection mandrel (PCL:PRGF). Both the PCL:PRGF(2-1) and PCL:PRGF scaffolds consisted of a 1:1 volume ratio of PCL:PRGF solution. Control scaffolds contained no PRGF.

Processing parameters varied with the polymer, while all solutions were electrospun onto a grounded rectangular stainless steel mandrel (1.9 cm wide×7.5 cm long×0.5 cm thick) rotating at 400 RPM and translating at 6 cm/s over a distance of 12 cm, using a Becton Dickinson syringe fitted with a blunt tip 18 gauge needle and a KD Scientific syringe pump. SF solutions were electrospun using a charging voltage of +25 kV, an air gap distance of 15 cm, and a flow rate of 4 ml/hr. PGA and PCL solutions were electrospun using a charging voltage of +22 kV, an air gap distance of 15 cm, and a flowrate of 6 ml/hr. PCL:PRGF(2-1) used a charging voltage of +30 kV placed on the end of the output nozzle, an air gap distance of 15 cm, and a flow rate of 2.5 ml/hr. PCL:PRGF used charging voltages of +25 and +27 kV for the PCL and PRGF solutions, respectively, an air gap distance of 15 cm from each syringe to the collecting mandrel, and a flow rate of 2.5 ml/hr for each solution.

### Characterization of Electrospun Structures

Dry, representative samples from each of the electrospun scaffolds was characterized through scanning electron microscopy (SEM, Zeiss EV050) to ensure the fibrous nature of the structures. Average fiber diameters and pore areas were calculated by taking 60 random fiber/pore measurements from across the SEM image using ImageTool 3.0 software (Shareware provided by UTHSCSA).

Uniaxial tensile testing was performed on a set of representative dog-bone shaped samples (overall length of 20 mm, 2.67 mm at its narrowest point, gage length of 7.5 mm, n=5) punched from each of the materials electrospun. All specimens were soaked for 4 hours in DI water prior to testing, with all SF samples first being soaked in ethanol for 30 minutes to promote beta sheet formation. Samples were then uniaxially tested to failure at a rate of 10 mm/min (1.33 min⁻¹ strain rate) using an MTS Bionix 200 testing system with a 100 N load cell (MTS Systems Corp.) Peak stress, modulus, and strain at break were calculated using TestWorks version 4.

### Evaluation of Cell Interaction

To determine the response of human cells on the PRGF containing scaffolds, 10 mm diameter disks from each electrospun scaffold were punched, disinfected (30 minute soak in ethanol followed by three 10 minute rinses in PBS), and placed in a 48-well plate. Each scaffold had a sterile Pyrex cloning ring (10 mm outer diameter, 8 mm inner diameter) placed on top to keep the scaffolds from floating, and to ensure that all cells stayed on the surface of the scaffold during culture. Each scaffold was then seeded with 50,000 human adipose derived stem cells (ADSC) in 500 μl of culture media (DMEM low glucose, 10% FBS, 1% penicillin/streptomycin, Invitrogen). Media was changed every third day, and samples were removed from culture and fixed in buffered formalin on days 7 and 21 for Hematoxylin and Eosin (H&E) staining.

### Quantification of Protein Release Kinetics

From each electrospun material 10 mm diameter disks were punched (n=3), disinfected (30 minute soak in ethanol followed by three 10 minute rinses in PBS), and placed in a 48-well plate with 500 μl of PBS. The PBS was changed and retained for evaluation on days 1, 4, 7, 10, 14, 21, 28, and 35. Samples at each time point were subjected to a generic protein assay (BCA Protein Assay, Thermo Scientific Pierce) to quantify the concentration of total protein released. Based upon these results, specific ELISAs were run on the retained samples (days 1, 4, 7, 10, 14, and 21) to detect proteins found in high concentrations in PRP, RANTES, PDGF-BB (Antigenix America Inc.), and TGF-β (Promega Corp.), to demonstrate that it was in fact PRGF being eluted from the scaffolds.

### Released PRGF Effect on Cell Proliferation

To determine the mitogenic potential of the PRGF released from the electrospun scaffolds on both ADSCs and macrophages, 10 mm diameter disks of each electrospun material were punched, disinfected (30 minute soak in ethanol followed by three 10 minute rinses in PBS), and placed in a 48-well plate (n=3). Each well was then seeded with 300,000 human peripheral blood macrophages (ATCC, CRL9855) in 500 μl MSFM, as well as in a control tissue culture polystyrene (TCPS) well containing MSFM with 1 mg/ml PRGF added (TCPS:PRGF). The macrophage conditioned media was removed daily and used as a supplement to feed ADSCs (200 μl MSFM with 300 μl ADSC media) cultured on TCPS (25,000 cells/well) in a separate 48-well plate. On days 1, 4, and 7 media was removed from the wells containing macrophages and replaced with 300 μl trypsin to remove macrophages for counting. After 5 minutes trypsin was deactivated with 300 μl MSFM, pipetted up and down gently several times, and the suspended macrophages were counted using an automated cell counter (Invitrogen Countess). ADSC proliferation was analyzed using an MTS Assay (Promega) at days 1, 4, and 7.

This study was then replicated without the use of macrophage conditioned media to isolate the impact of the released PRGF on ADSC proliferation. That is, disinfected 10 mm diameter disks of each electrospun material were placed in a 48-well plate with 500 μl ADSC culture media. Media was removed daily and used as a supplement to feed ADSCs cultured at 25,000 cells/well in a separate 48-well plate. ADSC proliferation was determined with an MTS Assay (Promega) at days 1, 4, and 7.

### Statistical Analysis

All statistical analysis was based on a Kruskal-Wallis one-way ANOVA on ranks and a Tukey-Kramer pairwise multiple comparison procedure (α=0.05) performed with the JMP®IN 8.0 statistical software package (SAS Institute, Inc.). Graphical depictions of mean data were constructed with Microsoft Excel 2007, with error bars representing standard deviations.

### Results

### Creation of PRP and PRGF

Based upon the platelet counts performed at the Harvard Immune Disease Institute's Blood Research laboratory, it was determined that the pooled whole blood used for this study contained 175×10³ platelets/μl, while the PRP created with the SmarPReP2 system yielded 955×10³ platelets/μl. This 5.5 fold increase in platelets is consistent with published data, and should result in a similar fold increase in growth factor concentration based upon the linear relationship between platelet and growth factor concentration.

The results of the bFGF and VEGF ELISAs (not shown) revealed that the FTF method of activation, essentially lysing platelets to release their a and dense granule contents, to be as effective, if not more so, than the traditional PRP activation techniques of thrombin and CaCl₂ for releasing growth factors. The FTF activation method resulted in average growth factor concentrations of 0.4 ng/ml for bFGF, and 1.6 ng/ml for VEGF. Using the traditional CaCl₂ and thrombin activation methods bFGF values were 0.8 and 0 ng/ml, respectively, while the VEGF values were 0.3 and 0.7 ng/ml, respectively. While there were few statistical differences between the different methods in the bFGF ELISA, with only the CaCl₂ activated different from the thrombin activated, the VEGF ELISA results demonstrated clearly that the FTF method was significantly greater than the other activation methods. It should be noted that the thrombin activation method resulted in an instantaneous gel, making it difficult to obtain liquid samples for ELISA analysis. The CaCl₂ activated PRP contained visible floating platelet aggregates, but was mostly liquidous, while the FTF activated PRP was completely liquid with no evidence of platelet aggregation.

### Chemotactic Effect of PRGF on Macrophages

The results of macrophage chemotaxis in response to a dose of PRGF dissolved in MSFM were investigated. The results showed that macrophage chemotaxis increased with the amount of PRGF until the concentration of 1 mg/ml, above which it became significantly reduced. While a trend was apparent, the only value that was significantly different from the group was the 1 mg/ml PRGF concentration, potentially indicating an ideal concentration for stimulating macrophage chemotaxis. It should be noted that the addition of powdered PRGF to MSFM resulted in a complete gel at 10 mg/ml, and a partial gel at 5 mg/ml. This resulting gelation may have had a negative impact on macrophage chemotaxis; however, it does indicate a reserve of active fibrinogen contained within the powdered PRGF capable of forming a clot in the presence of the Ca²⁺ found in the MSFM.

### Characterization of Electrospun Structures

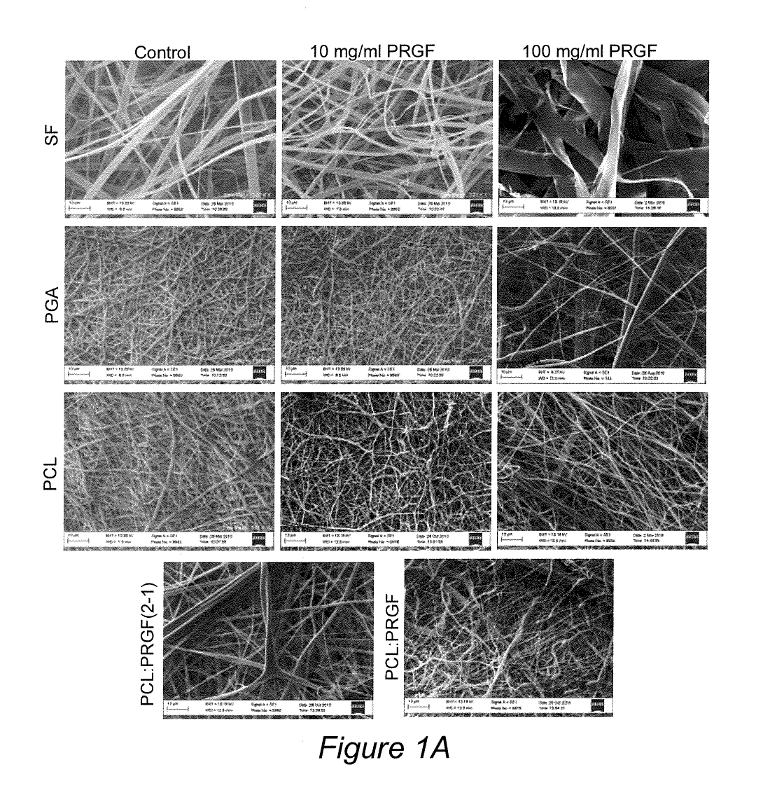

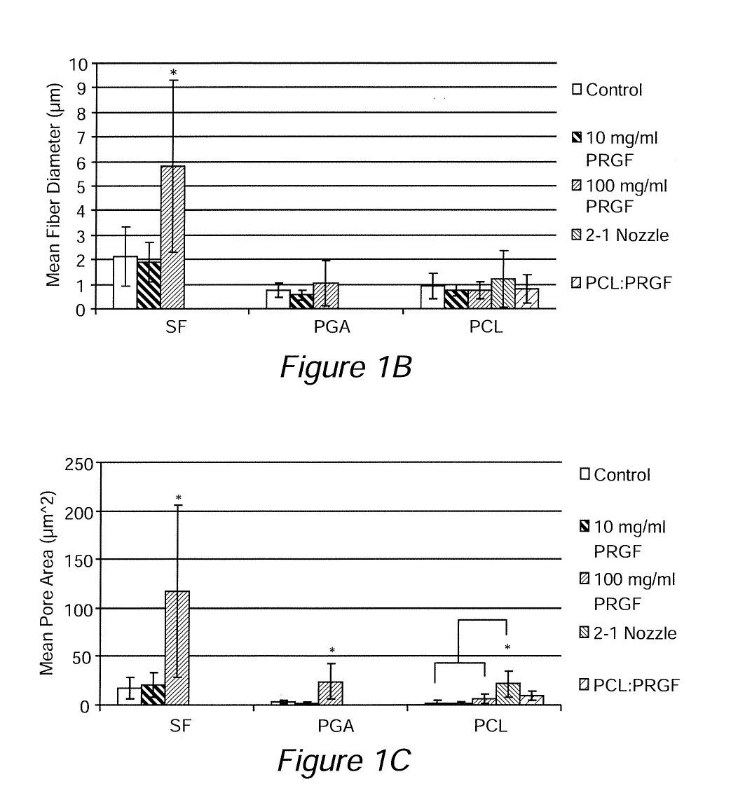

The results of the electrospun scaffold SEM characterization are shown in FIG. 1A-C. These SEMs demonstrate the fibrous nature of each of the electrospun scaffolds, both with and without PRGF. Mean fiber diameters for these scaffolds ranged from 0.5 μm for PGA:PRGF(10) to 5.8 μm for SF:PRGF(100). With the exception of the SF:PRGF(100), there were no significant differences in mean fiber diameter between the control scaffolds and the PRGF containing scaffolds. Somewhat surprisingly, the inclusion of PRGF had no real impact on the average size of the electrospun fibers, although it does appear that with the inclusion of high concentrations of PRGF and in the PCL:PRGF(2-1) and PCL:PRGF scaffolds there are a number of extremely small diameter fibers. Disregarding the SF scaffolds, this potential divergent population of PRGF fibers and synthetic polymer fibers may be evident through the rather large standards of deviation determined for those structures. Additionally, those same scaffolds appeared to exhibit an increase in void space visible in the SEMs as PRGF content was increased. Average pore areas (FIG. 1A-C) were found to correlate to average fiber diameters; as fiber diameter increased, pore area increased. This phenomenon has been well documented in previous electrospinning studies. The addition of 100 mg/ml PRGF to SF and PGA resulted in significantly increased pore areas over the control and 10 mg/ml PRGF containing samples, while the only differences seen in the PCL structures was between the PCL:PRGF(2-1) and the PCL:PRGF(100), PCL:PRGF(10), and PCL control scaffolds.

Mean peak stresses ranged from 0.2 MPa for PGA (100) to 5.2 MPa for PGA control, moduli ranged from 0.9 MPa for SF (100) to 21.4 MPa for PGA control, while average strain at break values ranged from 25.4% for PGA (100) to 112.8% for PGA control. In general, mechanical properties were shown to decrease significantly as PRGF concentration increased compared to values achieved for the PRGF-free control scaffolds. With the exception of the PCL:PRGF(10), average peak stresses and moduli were significantly lower for SF, PGA, and PCL scaffolds containing PRGF. These results were not unexpected, as traditionally the combination of biologic proteins in large concentrations (collagen, elastin, fibrinogen, etc.) with electrospun polymers regarded for their tensile strength typically results in significantly reduced mechanical strength.

The unique structures of PCL:PRGF(2-1) and PCL:PRGF, where both PRGF and PCL fibers were created, resulted in mechanical properties that fell between those of the PCL control and the PCL:PRGF(100) structures. This difference in mechanical properties appear to indicate that these blended scaffolds resulted in materials that were structurally different from those where PRGF was added directly to the electrospinning solution.

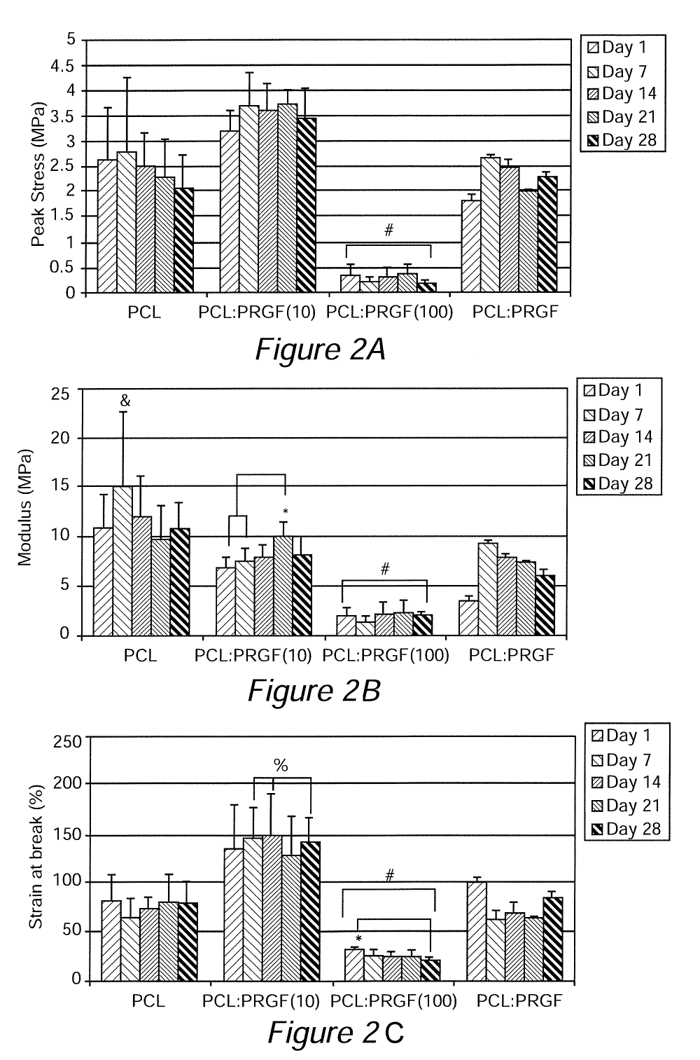

The results of the scaffold uniaxial tensile testing for selected constructs are shown in FIG. 2A-C. For these studies, dog-bone shaped samples (overall length of 20 mm, 2.67 mm at its narrowest point, gage length of 7.5 mm, n=5) were punched from each of the electrospun materials. All specimens were placed in a 6 well plate in complete media (DMEM low glucose supplemented with 10% FBS and 1% penicillin/streptomycin) and placed in an incubator (37° C., 5% CO₂) for 1, 7, 14, 21, or 28 days. Samples from each time point were then uniaxially tested to failure at a rate of 10 mm/min using an MTS Bionix 200 testing system with a 100 N load cell (MTS Systems Corp.). Peak stress, modulus, and strain at break were calculated using TestWorks version 4.

Results from the mechanical testing are shown in FIG. 2A-C, illustrating the ability for all scaffolds (with and without PRGF) to retain their mechanical strength over the 28 day time period. These results indicate there was little to no degradation occurring in these scaffolds: a surprising outcome considering the large amount of natural protein incorporated, especially in the PCL:PRGF(100) and PCL:PRGF scaffolds. Mechanical properties did decrease significantly in scaffolds of PCL:PRGF(100) compared to all other material types at all other time points.

### Evaluation of Cell Interaction

H&E staining was used to evaluate ADSC migration patterns on the scaffolds. H&E staining revealed confluent layers of ADSCs on the surfaces of the control scaffolds by day 7, while increased PRGF content resulted in increased cellular penetration into the scaffold. Surprisingly, after only 7 days ADSCs had migrated through half of the thickness of the PCL:PRGF(2-1) scaffold. By day 21 this trend was even more apparent, with clear cell migration through nearly the entire thickness of the PCL:PRGF(2-1) and PCL:PRGF scaffold (not shown). The SF:PRGF(100) scaffold also had nearly complete cellular penetration by day 21, compared to the SF scaffold containing no PRGF which exhibited only minimal migration into the depth of the structure. The PCL:PRGF(100) demonstrated a similar result, with the electrospun synthetic PCL material traditionally being difficult to cellularize in vitro, as it too exhibited increased cellular penetration when compared to the PCL scaffold containing no PRGF.

To determine the interaction of macrophages on electrospun PCL and PRGF incorporated scaffolds, 10 mm diameter discs were punched from the scaffolds, disinfected (30 minute soak in ethanol followed by three 10 minute rinses in PBS), and placed in a 48 well plate. Each scaffold was seeded with 50,000 murine peritoneal macrophages (ATCC, CRL TIB-186) in 500 μl complete media (RPMI 1640 supplemented with 10% FBS, 1% penicillin/streptomycin). Media was changed every third day, and on days 7 and 21, scaffolds were fixed in 10% Formalin and cryosectioned for 4′,6-diamidino-2-phenylindone (DAPI) staining.

In contrast to results obtained with ADSCs, results from DAPI staining of macrophages cultured on PCL and PRGF incorporated scaffolds revealed little cell penetration over 21 days. Macrophages appear to remain on the surface of all scaffolds, migrating the furthest into PCL scaffolds (75 μm) by 21 days.

### Quantification of Protein Release Kinetics

Quantified protein release from PRGF containing scaffolds was investigated. The results of this study demonstrated that scaffolds containing high concentrations of PRGF (SF:PRGF(100), PGA:PRGF(100), PCL:PRGF(100), and PCL:PRGF(2-1)) released detectable amounts of protein over 35 days in culture. The protein release from PRGF containing scaffolds peaked at day 1, decreased by about half on days 4 and 7, and reached a plateau that was sustained for the remainder of the duration. PCL:PRGF(2-1) scaffolds initially had the highest release of protein (300 μg/ml), but PCL:PRGF(100) had the highest release of protein at all time points after day 1 (125 μg/ml-50 μg/ml). Surprisingly, PCL:PRGF scaffolds released the lowest amount of protein over the 35 days, even though the concentration of PRGF incorporated was the same as that of PCL:PRGF(2-1) scaffolds. PGA:PRGF(100) and SF:PRGF(100) scaffolds had similar release kinetics as well, eliciting 240 μg/ml and 275 μg/ml of protein at day 1, respectively. Similar to the PCL:PRGF(100) and PCL:PRGF(2-1) structures, a plateau was achieved around 50 μg/ml and sustained until day 35. Minimal protein release was detected for PGA, SF, and PCL control scaffolds and scaffolds containing 10 mg/ml PRGF over the 35 days, indicating that the protein detected was in fact due to PRGF release and not simply an artifact of scaffold degradation.

Statistical analysis revealed protein release at day 1 from scaffolds of PGA:PRGF(100), SF:PRGF(100), PCL:PRGF(100) and PCL:PRGF(2-1) to be significantly greater than protein release from those respective scaffolds at all other time points (day 4-35). Additionally, scaffolds of PCL:PRGF(100) had significantly greater release at day 4 than day 35. The initial burst of release from the scaffolds at day 1 was expected as PRGF from the surface of the scaffolds was released. Remarkably, after the first day there was still a sustained release of protein from scaffolds of PGA:PRGF(100), SF:PRGF(100), PCL:PRGF(100) and PCL:PRGF(2-1) that continued throughout the 35 days, presumably due to the degradation of the polymer scaffolds and subsequent release of entrapped proteins.

Quantification of RANTES, PDGF-BB, and TGF-β from the PRGF containing scaffolds revealed detectable release over 21 days with kinetics similar to those of the protein assay results described previously. Scaffolds of PCL:PRGF(100) had the highest release of RANTES at day 1 (3 ng/ml), with a continual decrease in release thereafter. PGA:PRGF(100), SF:PRGF(100), and PCL:PRGF(2-1) exhibited a similar trend, with peak values of RANTES at day 1 of 2.5 ng/ml, 1.1 ng/ml, and 1 ng/ml for each scaffold, respectively. RANTES release from PCL:PRGF scaffolds had a peak of 0.5 ng/ml at day 1, but values were not detectable after day 4. Statistical analysis revealed RANTES release at day 1 from scaffolds of PGA:PRGF(100), SF:PRGF(100), PCL:PRGF(100) and PCL:PRGF(2-1) was significantly higher than release from those same scaffolds at all other time points (days 4-21). For PCL:PRGF(100), RANTES release at day 4 was significantly higher than that of all other time points for that scaffold.

PDGF-BB release was highest from scaffolds of PCL:PRGF(2-1), peaking at day 1 (0.3 ng/ml), and decreasing thereafter, with values not detectable after day 7. PDGF-BB was also detectable from scaffolds of PGA:PRGF(100), SF:PRGF(100) and PCL:PRGF(100), with the highest release occurring at day 1 (0.1 ng/ml, 0.075 ng/ml, and 0.15 ng/ml, respectively). PCL:PRGF scaffolds elicited PDGF-BB release of 0.03 ng/ml at day 1, but was undetectable thereafter. PDGF-BB release at day 1 from scaffolds of PGA:PRGF(100), PCL:PRGF(100) and PCL:PRGF(2-1) was significantly higher than release from those same scaffolds at all other time points (days 4-21). PDGF-BB release from SF:PRGF(100) at day 1 was significantly greater than release from the same scaffold at days 7-21. For PCL:PRGF(100) and PCL:PRGF(2-1), PDGF-BB release at day 4 was significantly higher than that of days 10-21 and all other time points with detectable values, respectively, for those scaffolds.

Much like the release of PDGF-BB from the scaffolds, TGF-β release was highest from scaffolds of PCL:PRGF(2-1). Peak release was seen on day 4 (1.17 ng/ml), although not significantly different from the release on day 1 (1.13 ng/ml), and decreased thereafter. Unlike RANTES and PDGF-BB, TGF-β release values were quantifiable for the PCL:PRGF(2-1), PCL:PRGF(100), and SF:PRGF(100) scaffolds throughout the 21 days evaluated. TGF-β release from scaffolds of PCL:PRGF(100) and PCL:PRGF(2-1) was significantly higher at days 1 and 4 than release from those same scaffolds at all other time points (days 7-21). In addition, release of TGF-β from scaffolds of PCL:PRGF(2-1) at days 7 and 10 was significantly higher than that at days 14 and 21. Surprisingly, PGA:PRGF(100) scaffolds did not exhibit a release above the minimum level of detection over the 21 days.

Similar to the protein assay results, RANTES, PDGF-BB, and TGF-β were undetectable from both the PGA, SF, and PCL control scaffolds and the scaffolds containing 10 mg/ml PRGF at all time points. The results of the statistical analysis illustrated that in general, after the initial release of growth factors from the surface of the scaffold at day 1, the release of RANTES and PDGF-BB that occurred at all time points thereafter is not significantly different, demonstrating a sustained release of growth factors from the scaffolds over the 21 day period as the polymer fibers begin to degrade. With regards to TGF-β, the PCL:PRGF(2-1) scaffolds exhibited a step-wise significant decrease in release until day 14, but still maintained a sustained quantifiable release.

### Released PRGF Effect on Cell Proliferation

The effect of PRGF release on macrophage proliferation was studied. As expected, macrophages proliferated in the presence of all scaffolds from days 1 and 4, however, by day 7 proliferation slowed, and in some cases, cell number even decreased. This may be due to nutrient levels insufficient to support the large number of cells in each well, and hence, resultant cell apoptosis. At day 1, there was no significant difference in macrophage number between the different scaffolds and TCPS. At day 4, there were significantly less macrophages on scaffolds of PCL:PRGF(2-1) than on scaffolds of SF, SF:PRGF(10), TCPS, and TCPS:PRGF, and may indicate a loss of macrophages due to cellular penetration into the highly bioactive PCL:PRGF(2-1) scaffolds. By day 7, there were no significant differences in macrophage proliferation on any scaffold. While these results indicated that in general, PRGF did not have an affect on macrophage proliferation, taken with the results from the prior macrophage chemotaxis study, it could instead be anticipated that PRGF promotes macrophage chemotaxis rather than proliferation.

To determine the role that PRGF had in the secretion of macrophage growth factors, ADSCs were cultured in media conditioned by macrophages exposed to released PRGF. The results of ADSC proliferation, when cultured in macrophage conditioned media, demonstrated no significant differences in proliferation at day 1. However, by day 4 ADSCs cultured in macrophage conditioned media from scaffolds of PCL:PRGF(100) and PCL:PRGF(2-1) had significantly greater proliferation than ADSCs cultured in macrophage conditioned media from the PCL and TCPS control, as well as all other scaffolds.

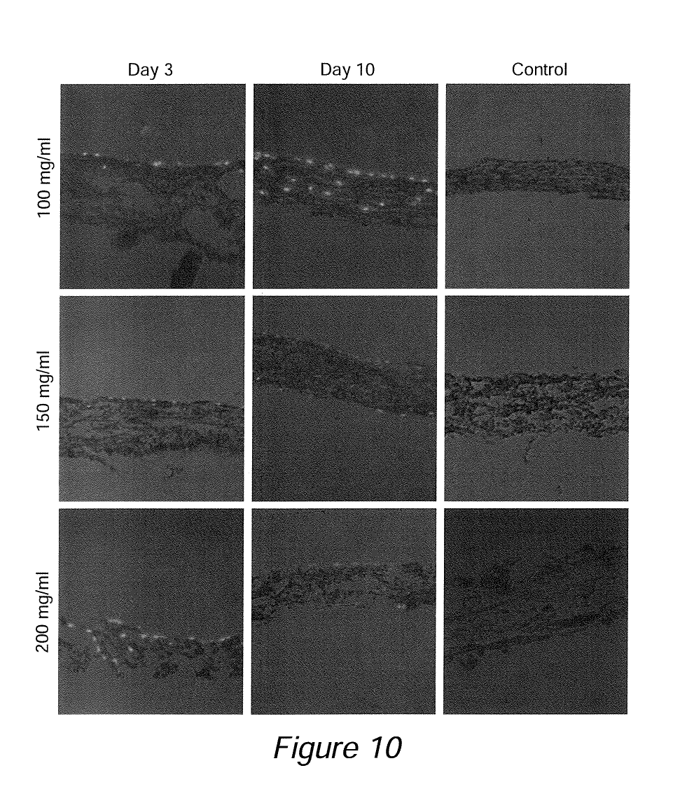

By day 7, there was significantly greater ADSC proliferation in macrophage conditioned media from scaffolds of PCL:PRGF(100), PCL:PRGF(2-1) and TCPS:PRGF than ADSCs cultured in conditioned media from PCL and TCPS control, as well as all other scaffolds. This was expected, as it had previously been demonstrated that PRGF, as well as growth factors secreted by macrophages, enhanced fibroblast, mesenchymal and stromal stem cell proliferation. In general, ADSC proliferation in all preconditioned media increased from day 1 to day 4, however, by day 7 it appeared that proliferation slowed, and in some cases cell number even decreased, potentially due to induced contact inhibition as the cells became confluent in the wells, or died off following exhaustion of media nutrients. This may also have been due to the fact that the conditioned media used for the ADSCs was macrophage serum free media, which is unfavorable over the long-term for ADSC growth, or due to harmful factors expressed during macrophage apoptosis. From the results in FIG. 10, it was evident that the effect of macrophages on ADSC proliferation was due to macrophage interaction with PRGF containing scaffolds, and not the number of macrophages.

ADSC proliferation when cultured in PRGF conditioned media without macrophages was investigated. Overall, ADSCs proliferated from day 1 to day 4 (with a few exceptions), and from day 4 to day 7, as expected. After 1 day, there were no significant differences in ADSC proliferation for any scaffold. By day 4, ADSCs cultured in media from scaffolds of SF:PRGF(100) had significantly greater proliferation than those cultured in media from SF control scaffolds. At day 7, ADSCs cultured in media from scaffolds of SF:PRGF(100) and PCL:PRGF(2-1) had significantly greater proliferation than cells cultured in media from SF and PCL control scaffolds, respectively. Compared to ADSCs cultured in media from the TCPS control, cells cultured in media from PCL:PRGF(2-1) and PCL:PRGF scaffolds had significantly greater proliferation at day 7. These results suggest that the presence of PRGF does impact ADSC proliferation, and corroborates previously published work. It is clearly evident from these studies that the proliferation of ADSCs, cultured in conditioned media, is different depending on the presence or absence of macrophages and macrophage secreted factors over the 7 day study duration, and will be discussed further in the following section.

### Chemotactic Effect of PRGF Incorporated Scaffolds on Macrophages

The effect of PRGF incorporated scaffolds on macrophage chemotaxis was investigated. Briefly, 10 mm diameter discs were punched from electrospun scaffolds incorporated with and without PRGF, disinfected (30 minute soak in ethanol followed by three 10 minute rinses in PBS), and placed in the bottom of a 24-well Transwell plate (8 μm diameter pores, Corning, Inc.). 600 μl of media (RPMI 1640 supplemented with 10% FBS, 1% penicillin/streptomycin) was placed in the bottom wells, while the top insert was seeded with 50,000 murine peritoneal macrophages (ATCC, TIB-186) in 200 μl of media for 72 hours (n=3). Following the 72 hour duration, the bottom wells were aspirated and an MTS assay was performed to determine the average cell number in each well.

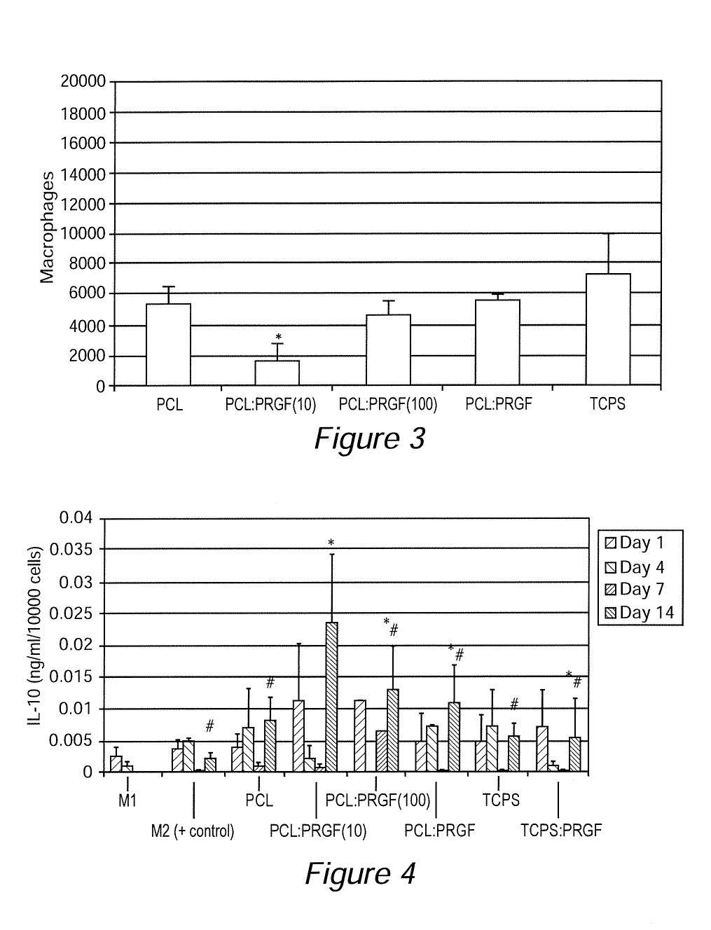

The results of macrophage chemotaxis in response to PRGF incorporated scaffolds are shown in FIG. 3. PRGF incorporated scaffolds did not have an effect on macrophage chemotaxis over the 72 hour period, and surprisingly, PCL:PRGF(10) scaffolds had a significantly reduced amount of macrophages.

Chemokine Release from Macrophages to Determine Phenotype

The chemokine release from macrophages cultured on the PRGF containing scaffolds was investigated. 10 mm diameter discs were punched, disinfected (30 minute soak in ethanol followed by three 10 minute rinses in PBS), and placed in a 48-well plate. Each scaffold was then seeded with 50,000 murine peritoneal macrophages in 500 μl culture media (RPMI 1640 supplemented with 10% FBS, 1% penicillin/streptomycin). In addition, macrophages were also seeded on tissue culture plastic (TCPS) without and with 1 mg/ml PRGF (TCPS:PRGF). For positive controls, macrophages were cultured on TCPS in media containing 100 ng/ml lipopolysaccharide (LPS, Sigma-Aldrich) plus 20 ng/ml IFN-γ (PeproTech, for M1 pro-inflammatory, polarization) or 20 ng/ml IL-4 (PeproTech, for M2, pro-regenerative, polarization). Macrophages were cultured in standard conditions (37° C., 5% CO₂) and supernatant was collected on days 1, 4, 7, and 14. Simultaneously, an MTS assay was performed to determine macrophage number on days 1, 4, 7, and 14. TNF-α (Antigenix America, Inc.) and IL-10 (PeproTech) ELISAs were performed per manufacturer's protocol to quantify the amount M1 (pro-inflammatory, TNF-α) and M2 (pro-regenerative, IL-10) chemokine being released. These results were normalized to amount of chemokine released (ng/ml) per 10,000 cells.

FIG. 4 shows the results of IL-10 release from macrophages cultured on PCL and PRGF incorporated scaffolds and TCPS with and without 1 mg/ml PRGF. At days 1, 4, and 7 there are no significant differences between the different scaffold types and control groups. However, by day 14, scaffolds and TCPS with increased amounts of PRGF (PCL:PRGF(10), PCL:PRGF(100), PCL:PRGF, and TCPS:PRGF) elicit significantly higher IL-10 release from macrophages over other control groups and scaffolds without PRGF. Another point worth noting is that, in general, IL-10 release is higher from macrophages cultured on electrospun scaffold materials than it is for the positive control (M2). These results indicate PRGF, as well as electrospun structures overall, have the ability to enhance the release of IL-10 from macrophages, possibly driving them towards the M2 phenotype.

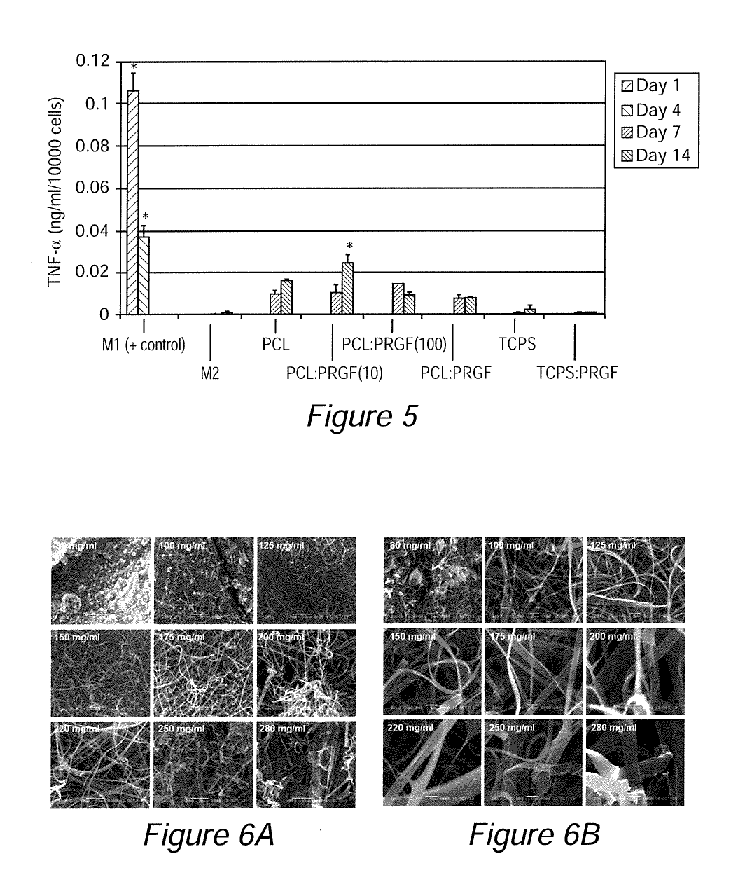

FIG. 5 shows the results of TNF-α release from macrophages. At days 1 and 4, only the positive M1 control has detectable TNF-α release. By days 7 and 14, release on all scaffold types is detectable, with statistical significance on PCL:PRGF(10) scaffolds at day 14. Although there is no release from the positive M1 control at days 7 and 14 (due to cell apoptosis), the amount of TNF-α release on electrospun scaffolds at days 7 and 14 is much lower than that from the positive control at days 1 and 4. Also worth noting is that at day 14, TNF-α release from macrophages cultured on all scaffold types is less than that of IL-10 release at day 14 (except for PCL). These results indicate PRGF, and electrospun structures overall, do not enhance the release of TNF-α (a pro-inflammatory chemokine) from macrophages over the positive control, and may be driving more cells towards the M2 phenotype over the M1 phenotype (as indicated by amount of chemokine released per 10,000 cells).

### Discussion

This present study provides a proof-of-principle for the incorporation of a powdered PRGF derived from human PRP into electrospun scaffolds of a number of materials. Through a number of evaluation methods, we were able to demonstrate that PRGF retained its physiologic activity after lyophilization and through the electrospinning process, subsequently enhancing the bioactivity of the electrospun scaffolds.

The use of PRP in clinical applications has been gaining in popularity as a means to stimulate tissue repair and regeneration with very minimal risk to the patient. However, the “black box” approach taken by many of the clinicians utilizing PRP leaves much to be done in the realm of basic science to fully understand and standardize the practice. To date, the collection of whole blood and the concentration and isolation of platelets to make PRP has been proven effective in vitro for stimulating cellular activity in a number of formats, both in liquid and in lyophilized PRGF form (Pietramaggiori et al., 2006; Pietramaggiori et al., 2007; Pietramaggiori et al., 2008; Sum et al. 2007).

To the best of the authors' knowledge, this disclosure serves as the first instance of a powdered PRGF being incorporated into an electrospun tissue engineering scaffold to serve as a controlled release vehicle for such a concentrated growth factor and cytokine milieu. While electrospun scaffolds have been used as growth factor delivery systems in the past [Sahoo et al., J Biomed Mater Res A 2010; 93(4):1539-50; Sahoo et al., Biomaterials 2010; 31(11):2990-8; Uebersax et al., Tissue Eng Part B Rev 2009; 15(3):263-89; Chew et al., Biomacromolecules 2005; 6(4):2017-2024; Zhang et al., Adv Drug Deliv Rev 2007; 59(4-5):360-73; and Chung et al., Adv Drug Deliv Rev 2007; 59(4-5):249-6] they have typically been limited to the incorporation of only a small number of growth factors due in part to the cost associated with purchasing the recombinant or isolated proteins (Uebersax et al., 2009). The incorporation of a cost-effective PRGF protein array into an electrospun structure has the potential to deliver a multitude of growth factors, cytokines, and chemokines in physiologically relevant ratios. Such a platelet-based growth factor cocktail would essentially replicate the necessary factors found in a site of normal wound healing and promote the formation of healthy tissue through the stimulation of the healing cascade. The results presented in this manuscript demonstrated the potential of such a sustained release vehicle through enhanced cellular activity consistent with other in vitro studies of PRP/PRGF.

Cellular migration and penetration into electrospun scaffolds was enhanced, regardless of polymer, with the addition of PRGF. While historically the ability for cells to migrate into an electrospun structure has been viewed as a challenge, especially with synthetic polymers such as PGA and PCL, the inclusion of PRGF yielded structures that were readily infiltrated. Without being bound by theory, it is possible that: 1) the presence of an array of chemotactic proteins found in large quantities in PRP may be the most logical explanation; or 2) it may also be an affect of the change in scaffold mechanical properties since scaffolds with higher PRGF content exhibited decreased mechanical properties which may have allowed for cellular migration into the scaffold to occur more readily. In addition, the presence of PRGF fibers intermingled amongst the polymer fibers of the scaffolds, particularly in the case of the PCL:PRGF(2-1) and PCL:PRGF structures may have also provided paths of easy entry into the thicknesses of the structures. These fibers of varying diameter had an apparent impact on the porosity of the scaffolds. As PRGF content increased, there was an increase in fiber diameter/pore area, which may have allowed for more rapid cellular infiltration while decreasing mechanical properties.

While the significant decrease in scaffold mechanical properties observed with the addition of large quantities of PRGF makes these scaffolds less than ideal for use in load-bearing tissue engineering applications, it was not completely surprising nor was it seen as a negative result. As previously mentioned, this decrease in individual fiber mechanical properties may have contributed to the rapid cellular infiltration of the scaffolds. This enhanced cellular infiltration, regardless of its root cause, would allow for a tissue engineered product to be more rapidly remodeled with native collagen extracellular matrix; the production of which would readily supplement the strength of the scaffolds and encourage incorporation into surrounding tissues. Many factors found in PRGF have been proven to increase collagen matrix production in a number of cell types. TGF-β, one such well-known matrix production related growth factor, was released by the electrospun scaffolds in detectable quantities, in some cases for up to 21 days, likely accelerating matrix production, and improving the mechanical strength of the weakened PRGF containing scaffolds.

The loss of mechanical strength of PRGF fibers within the electrospun scaffolds containing high concentrations of PRGF (SF:PRGF(100), PGA:PRGF(100), PCL:PRGF(100), PCL:PRGF(2-1), and PCL:PRGF) suggest that the PRGF fibers are best utilized in a role of enhancing scaffold bioactivity rather than load bearing.

As demonstrated in the protein release assays conducted herein, detectable levels of proteins were released from the electrospun scaffolds for up to 35 days in vitro. The fact that RANTES, PDGF-BB, and TGF-β were detectable in specific materials at up to 21 days attests to the sustained release nature of the structures. The release of RANTES, PDGF-BB, and TGF-β release was analyzed as they are three of the more highly concentrated proteins contained within PRP/PRGF. However, from these results it can be interpolated that other factors such as PDGF-ab, FGF, and EGF were released in the same fashion. The nature of the release may be effective in enhancing migration of cells from surrounding tissues, with a large burst of protein creating a substantial chemotactic gradient, followed by a sustained release of protein to promote cell proliferation, and scaffold infiltration and remodeling. The incorporation and subsequent release of albumin may in fact serve as a protectant for the cytokines and chemokines included in the PRGF. The hydrophilic albumin molecules have been demonstrated in the literature to have the potential to encapsulate smaller proteins, and effectively shield them from potential denaturation.

The retention of PRGF's biological activity following the electrospinning process, subjected to both high voltages and the organic solvent HFP, is displayed in the ADSC response to conditioned media from PRGF containing scaffolds cultured with and without macrophages. In both cases, ADSC proliferation was enhanced over 7 days when cultured in conditioned media from scaffolds containing PRGF versus those not containing PRGF. The presence of several growth factors within PRGF known to induce cell proliferation (VEGF, PDGF, IGF, FGF, and EGF, etc.) is most likely the reason for enhanced ADSC proliferation.

ADSC proliferation may be due, in part, to the various growth factors and cytokines secreted by the macrophages. It has been shown previously that macrophages cultured on electrospun scaffolds have the ability to produce high levels of VEGF and FGF, and in the presence of PRGF, produce additional pro-angiogenic growth factors and cytokines, including those which enhance cell proliferation [Garg, K.; Sell, S. A.; Madurantakam, P. and G. L. Bowlin. Angiogenic Potential of Human Macrophages on Electrospun Bioresorbable Vascular Grafts. Biomedical Materials, 4(3), 31001 (Epub Apr. 17, 2009), 2009].

In conclusion, this study demonstrated the potential for PRP to be subjected to a FTF process, lyophilized to create PRGF, and incorporated into electrospun scaffolds of various materials. This PRGF was released from the electrospun scaffolds in a controlled fashion over a period of 35 days in culture, and retained its potential to positively influence the proliferation of ADSCs and chemotaxis of macrophages at specific concentrations in vitro. Additionally, the presence of PRGF in high concentrations allowed for the rapid infiltration of ADSCs into electrospun structures of both natural and synthetic polymers when cultured in vitro for 21 days. As one of the major advantages of PRP, when used clinically, is its ability to deliver a milieu of growth factors and cytokines at the patients' bedside, the creation of an off-the-shelf electrospun scaffold incorporating PRGF from pooled allogenic blood may have the same benefits. While the use of pooled blood is typically frowned upon in the United States, the use of allogenic PRP has been gaining popularity in a number of European studies with no mention of adverse immune reactions.

### Example 2

### The Creation of Electrospun Nanofibers from Platelet Rich Plasma

### Abstract.

A “preparation rich in growth factors” (PRGF), contains supraphysiologic amounts of autologous growth factors and cytokines known to enhance wound healing and tissue regeneration. This example reports the first results of electrospinning nanofibers from PRGF to create fibrous scaffolds that could be used for various tissue engineering applications. Human platelet rich plasma (PRP) was created, activated by a freeze-thaw-freeze process, and lyophilized to form a powdered preparation rich in growth factors (PRGF). It was dissolved in 1,1,1,3,3,3-hexafluoro-2-propanol (HFP) at different concentrations to form fibers with average diameters of 0.3-3.6 μm. A sustained release of protein from the PRGF scaffolds was demonstrated up to 35 days, and cell interactions with the PRGF scaffolds confirmed cell infiltration after just 3 days. As electrospinning is a relatively simple process, and PRGF contains naturally occurring growth factors in physiologic ratios, creating nanofibrous structures from PRGF has the potential to be beneficial for a variety of tissue engineering applications.

### 1. Introduction

The creation of tissue engineering scaffolds through the process of electrospinning has yielded promising results for the field of regenerative medicine over the last decade. These scaffolds can replicate the sub-micron scale topography of the native extracellular matrix (ECM), through the creation of nanoscale, non-woven fibers, using a number of natural and synthetic polymers. Using the process of electrospinning it is also possible to control the alignment and orientation of the created fibers, creating scaffolds that can be easily customized for nearly any tissue in the body. This control over fiber orientation, coupled with the diverse array of polymers conducive to being electrospun, allows for the tissue engineer to create structures with tailorable mechanical properties. Additionally, these scaffolds exhibit high surface area-to-volume ratios, high porosities, and variable pore-size distributions that mimic the native ECM and effectively create a dynamic structure capable of sustaining the passive transport of nutrients and waste throughout the structures.

However, despite the porosity and flexibility afforded by the electrospinning process, it is still considered to be quite challenging to promote cellular penetration into the depth of an electrospun structure, with cells preferring to proliferate and migrate across the surface of the scaffold rather than venture inside it. While a number of rather novel techniques have been employed to increase cellularization of electrospun scaffolds, nothing has been proven to be ideal, nor to date become common practice in the field. The incorporation of growth factors into electrospun matrices for tissue engineering has the potential to enhance scaffold bioactivity, by supplying appropriate physical and chemical cues to promote cellular proliferation and migration, thereby increasing the cellularization of the structures. By replicating the role of the native ECM in the normal wound healing cascade, that is serving as a reservoir of soluble growth factors critical to regeneration and providing a template for tissue repair, it may be possible to accelerate cellularization and tissue repair.

Platelet-rich plasma (PRP) is a supraphysiologic concentration of platelets suspended in plasma intended to serve as an autologous source of concentrated growth factors and cytokines. The use of PRP has been growing rapidly in the clinical world, as activated-PRP (aPRP) has been proven effective in accelerating healing in a number of tissues: osteochondral defects, tendon/ligament injuries, and chronic skin wounds (diabetic and pressure ulcers). The creation of aPRP is a relatively simple procedure that can be performed bedside, typically involving a blood draw and centrifugation to concentrate the platelet portion, followed by a platelet activation step and the delivery of the aPRP to the site of injury. There have been several methods reported in the literature on successfully activating and delivering aPRP to an injury site, with most involving the creation of a platelet gel using thrombin or CaCl₂. These aPRP gels can then be easily applied to wound sites through injection or topical application.

The basis behind the use of these aPRP gels is that through the activation of the platelets, the alpha and dense granules contained within the platelets release an array of growth factors and cytokines critical to mediating normal wound healing. The milieu of growth factors and cytokines released from the aPRP are in physiologically relevant ratios, albeit in concentrations several times higher than that of normal blood due to the linear relationship between platelet and growth factors concentrations. Activated PRP has been shown to contain platelet derived growth factor (PDGF), transforming growth factor-0 (TGF-(3), vascular endothelial growth factor (VEGF), fibroblast growth factor (FGF), epidermal growth factor (EGF), and others in elevated concentrataions. Activated PRP has also been shown to contain a number of macrophage and monocyte mediators such as RANTES (Regulated upon Activation, Normal T-cell Expressed, and Secreted), lipoxin, and an array of interleukins capable of mediating inflammation. In addition, the plasma portion of the PRP contains the proteins albumin, fibrinogen, a number of immunoglobulins, and more.

The majority of previous studies have investigated methods of delivering single or multiple growth factors from isolated or recombinant sources. However, such methods can be prohibitively expensive and fail to deliver physiologically relevant concentrations. The purpose of this study was to create an electrospun scaffold that would harness the reparative potential and bioactivity found in aPRP, namely the growth factor and cytokine milieu contained within, by lyophilizing aPRP and creating PRGF suitable for electrospinning. Utilizing the plasma proteins contained within the PRGF, namely fibrinogen which has been successfully electrospun in the past, it was hypothesized that pure lyophilized PRGF could be electrospun into a stable scaffolding material for tissue engineering applications. Such a scaffold, containing a concentration of multiple growth factors and cytokines, would have the potential to promote cellularization of the structure while providing a sustained release of growth factors capable of providing a chemotactic gradient for cellular recruitment.

### 2. Methods

2.1 Creation of aPRP and PRGF

Fresh human whole blood from 3 donors was purchased (Biological Specialty Corp., Colmar, Pa., USA), pooled, and used in a SmartPReP® 2 (Harvest Technologies Corp., Plymouth, Mass., USA) centrifugation system to create PRP per manufacturers protocol. A small aliquot of both pooled whole blood and PRP were sent to the Harvard Immune Disease Institute's Blood Research laboratory to determine their respective platelet concentrations. PRP was then subjected to a freeze-thaw-freeze (FTF) cycle for platelet lysis and activation. Briefly, PRP was placed in a −70° C. freezer for 24 hrs followed by a 37° C. waterbath for 1 hr, and then returned to the −70° C. freezer for 24 hrs. This method has previously been found to contain the same, and in some cases, higher levels of bFGF and VEGF as thrombin and CaCl₂ aPRP (data not published). Frozen aPRP was then lyophilized for 24 hrs to create a dry PRGF powder which was finely ground in a mortar and pestle prior to use.

### 2.2 Creation of Electrospun PRGF Scaffolds

PRGF was dissolved in 1,1,1,3,3,3-hexafluoro-2-propanol (HFP, TCI America Inc., Portland, Oreg., USA) at different concentrations, ranging from 80-280 mg/ml, to determine the optimum fiber forming concentration range. HFP was used as the solvent because, not only is it the most widely used solvent in our lab, but it also is very versatile in creating nanofibrous scaffolds from a variety of natural and synthetic polymers without much difficulty. In addition, previously published studies performed by our lab, as well as many others, have shown electrospun scaffolds fabricated from HFP are biocompatible. Once in solution, PRGF was loaded into a 3 mL Becton Dickinson syringe, and placed in a KD Scientific syringe pump (model number 100, Holliston, Mass., USA) for dispensing at a rate of 2.5 ml/hr. A blunt 18 gauge needle was placed on the syringe, and the positive voltage lead of a power supply (Spellman CZE1000R; Spellman High Voltage Electronics Corp., Hauppauge, N.Y., USA) was attached to the needle and set to 25 kV. A grounded mandrel (1.9 cm wide×7.6 cm long×0.5 cm thick; 303 stainless steel) was placed 15 cm away from the needle tip and was rotated at 500 rpm and translated at 7.5 cm/s over 15 cm distance for collection of the fibers.

### 2.3 Electrospun Scaffold Characterization

### 2.3.1 Scanning Electron Micrographs and Fiber Diameter.

Fiber diameter characterization was accomplished using scanning electron micrographs (SEM, Zeiss EVO 50 XVP, Peabody, Mass., USA) of each scaffold. Samples from each scaffold were mounted on an aluminum stub and sputter coated with gold for imaging. The average fiber diameter of each electrospun structure was determined from the SEM images using UTHSCSA ImageTool 3.0 software (Shareware provided by University of Texas Health Science Center in San Antonio). Fiber diameter averages and standard deviations were calculated by taking the average of 60 random measurements per micrograph.

### 2.3.2 Protein Release Kinetics.

From scaffolds of 100, 150, and 200 mg/ml PRGF, 10 mm diameter discs were punched, disinfected with a 30 minute soak in ethanol, followed by three 10 minute rinses in PBS, and placed in a 48 well plate with 500 μl of PBS. On days 1, 4, 7, 10, 14, 21, 28 and 35, PBS was retained and kept in a −70° C. until ready for evaluation. A generic protein assay (BCA Protein Assay, Thermo Scientific Pierce, Rockford, Ill., USA) was performed on samples to quantify the concentration of total protein released from the PRGF scaffolds.

### 2.3.3 Gel Electrophoresis.

Gel electrophoresis was performed to analyze the molecular weight of the proteins in PRGF, platelet poor plasma (PPP), and electrospun PRGF scaffolds and compare them to those of fibrinogen (FBG, Sigma Aldrich, St. Louis, Mo., USA) and bovine serum albumin (BSA, Sigma Aldrich, St. Louis, Mo., USA) controls. Briefly, 2 mg BSA, FBG, PRGF, and 100, 150, 200 mg/ml PRGF scaffolds were solubilized in a reducing agent containing laemmeli buffer with 5% (3-mercaptoethanol. Samples were boiled for 3-5 minutes to further ensure they were solubilized, and 10 μL of each sample was placed in duplicate in each lane of 4-15% polyacrylamide 18-well gels (Criterion Bio-Rad, Hercules, Calif., USA). A molecular weight protein ladder (20 μL, Sigma Aldrich, St. Louis, Mo., USA) was run to provide a molecular weight basis for protein identification and comparison. Samples were run at constant voltage of 120 V over 2 hours. After the 2 hours, the gels were stained with Coomassie Blue, and evaluated by the Bio-Rad Gel Doc™ 2000 system.

### 2.3.4 Fluorescent Based Assay.

FBG concentration was quantified in the PRGF electrospun scaffolds, as well as in aPRP, blood and PPP by using a fluorescent based assay. Scaffolds of 100, 150, and 200 mg/ml electrospun PRGF (10 mm diameter discs, n=4) were placed in a 48 well plate and blocked with Odyssey Blocking Buffer (LI-COR Biosciences, Lincoln, Nebr., USA) for 1 hour at room temperature. Simultaneously, human FBG from reference plasma (Fisher Scientific, Pittsburgh, Pa., USA) was diluted in DI water at concentrations of 76, 38, 19, 9.5, 4.75, 2.38, 1.19, 0.59, and 0 mg/dl and was blotted on a PVDF membrane, along with aPRP, PPP, blood, and PRGF diluted in water at 10, 5, and 1 mg/ml. The membrane was blocked in Odyssey Blocking Buffer for one hour at room temperature. After blocking, standards and samples were incubated in anti-human fibrinogen antibody (Millipore, Billerica, Mass., USA) at room temperature for 1.5 hours. All samples and standards were then washed four times with 0.1% Tween-20 in PBS, after which the signal from mouse anti-human fibrinogen antibody was detected with goat anti-mouse IgG secondary antibody tagged with a fluorescent 800 nm marker (ThermoScientific, Pittsburgh, Pa., USA). To account for antibody background fluorescence, each scaffold was incubated with secondary antibody only. Samples and standards were incubated in the secondary antibody for 1 hour at room temperature without exposure to light. After washing, the samples were scanned using the 800 nm channel of the Odyssey Infrared Imaging System (LI-COR Biosciences, Lincoln, Nebr.) at an intensity of 3.5. Fluorescence intensities were measured using circular gates that completely surrounded the scaffolds and well plates. Background fluorescence that was obtained from samples incubated with secondary antibody only was subtracted from the signal intensities of the samples incubated with both primary and secondary antibodies.

### 2.4 Evaluation of Cell Interaction

To determine the interaction of human cells on electrospun PRGF scaffolds, 10 mm diameter discs were punched from scaffolds of 100, 150, and 200 mg/ml PRGF, disinfected (30 minute soak in ethanol followed by three 10 minute rinses in PBS), and placed in a 48 well plate. A sterile Pyrex cloning ring (10 mm outer diameter, 8 mm inner diameter) was placed on top of each scaffold to prevent them from floating, and to ensure all cells stayed on the surface of the scaffold during culture. Each scaffold was seeded with either 100,000 human adipose derived stem cells (hADSC) isolated from medical waste lipoaspirate [43] in 500 μl of complete media (DMEM low glucose supplemented with 10% FBS and 1% penicillin/streptomycin, Invitrogen, Carlsbad, Calif., USA) or 100,000 human umbilical artery smooth muscle cells (hSMC, Lonza, Basel, Switzerland) in 500 μl of complete media (SMGM-2 bullet kit, Lonza, Basel, Switzerland). Controls consisted of scaffolds in complete media without cells. Media was changed every third day, and on days 3 and 10, scaffolds were fixed in 10% Formalin and cryosectioned for 4′,6-diamidino-2-phenylindole (DAPI) staining.

### 2.5 Statistical Analysis

Statistical analysis was performed using JMP®IN 8.0 statistical software (SAS Institute, Inc., Cary, N.C., USA) and was based on a Kruskal-Wallis one-way analysis of variance on ranks and a Tukey-Kramer pairwise multiple comparison procedure (α=0.5). Graphical depictions of mean data were constructed with Microsoft Excel 2007, with error bars representing standard deviations.

### 3. Results and Discussion

3.1 Creation of aPRP and PRGF

It was determined by Harvard Immune Disease Institute's Blood Research Laboratory that the PRP created by the SmartPReP® 2 centrifugation system resulted in a 5.5 fold increase in platelets compared to the pooled whole blood used in this study (955×10³ platelets/μl versus 175×10³ platelets/μl). This result is in agreement with published data, and based upon the linear relationship between platelet count and growth factor concentration, a resulting similar fold increase should present in platelet derived growth factor concentrations.

### 3.2 Electrospun Scaffold Characterization

### 3.2.1 SEM and Fiber Diameter.

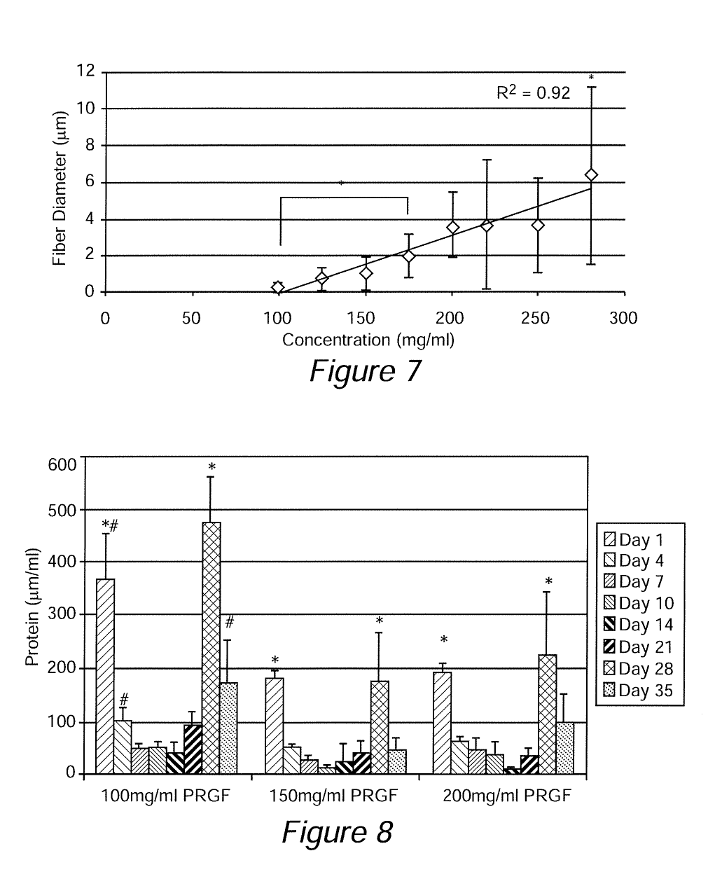

SEM characterization of the electrospun PRGF scaffolds is shown in FIG. 6. The micrographs illustrate the nanofibrous structures of each scaffold starting around 100 mg/ml, with the appearance of a large range of fiber diameters for the different PRGF concentrations, as well as increased void space as PRGF concentration increases. Fiber diameters for the scaffolds range from 0.28±0.29 μm for 100 mg/ml PRGF to 6.37±4.81 μm for 280 mg/ml PRGF (FIG. 7). Statistical analysis reveals the average fiber diameters for 100 and 175 mg/ml PRGF are significantly different from all other scaffolds, with the exception of 125 and 150 mg/ml PRGF. Average fiber diameters for scaffolds of 200, 220, and 250 mg/ml PRGF are significantly different from all other scaffolds, but not each other. Scaffolds of 280 mg/ml PRGF have significantly greater fiber diameters from those of all other scaffolds. This linear relationship between polymer concentration and fiber diameter is expected, as it has been well established previously. The broad range of fiber diameters produced during the electrospinning process allows for flexibility in the fabrication of electrospun scaffolds for different tissue engineering applications.

### 3.2.2 Protein Release Kinetics.

The quantified protein release results from electrospun PRGF scaffolds are shown in FIG. 8, demonstrating protein release from PRGF scaffolds was detectable over 35 days. The release kinetics illustrates a peak release of protein on day 1 for all scaffold concentrations, followed by a distinct decrease in release for days 4-21. Unexpectedly, at day 28, protein release increases for all scaffolds. By day 35 protein release has decreased, however, compared to days 4-21, overall release has increased. Surprisingly, 100 mg/ml PRGF scaffolds had the highest release of protein over the 35 days compared to the other scaffolds, releasing 370 μg/ml of protein at day 1 and 175 μg/ml by day 35. Scaffolds of 150 and 200 mg/ml PRGF had similar release kinetics over the 35 days (185-49 μg/ml and 194-96 μg/ml for the 150 and 200 mg/ml PRGF scaffolds, respectively). The initial burst release from scaffolds at day 1 is expected, as PRGF from the surface of the scaffolds is released. The high protein release from scaffolds of 100 mg/ml PRGF over the other scaffolds may be explained by the scaffold's smaller fiber diameters. Although not specifically investigated in this study, other previously published studies have demonstrated similar results, and explain that with smaller fiber diameters there is less distance for molecules to traverse to reach the fiber surface, hence, more protein release from the fibers over time. The rise in protein release after day 21 may be due to fiber degradation of the PRGF scaffolds occurring around 28-35 days and subsequent release of entrapped proteins. Although degradation may have started to occur around day 28, the electrospun scaffolds were still very much intact at 35 days. This outcome was unexpected, as most electrospun natural polymers degrade rapidly in solution and need to be cross-linked or co-spun with a synthetic polymer to increase their stability. Statistical analysis revealed protein release at day 1 and day 28 from all PRGF scaffolds was significantly greater than protein release from those respective scaffolds at all other time points (days 4, 7, 10, 14, 21, and 35). For scaffolds of 100 mg/ml PRGF, protein release at days 1, 4, and 35 was significantly different from that at all other time points for that respective scaffold (days 7, 10, 14, and 21).

### 3.2.3 Gel Electrophoresis.

Electrophoretic patterns of molecular weight standards, FBG, PRGF, PPP, PRGF scaffolds of 100, 150, and 200 mg/ml and BSA were determined using 4-20% polyacrylamide gels (not shown) The electrophoretic pattern of FBG appeared as expected, as it has been previously determined that the alpha, beta and gamma chains have average molecular weights of around 68, 58 and 50 kDa, respectively. BSA also exhibited a pattern that would be expected, with a distinct band around 67 kDa. The electrophoresis results of PRGF resembled those of BSA and the FBG alpha chain, with a distinct band around 68 kDa. This band is also likely to be representative of hemoglobin, which has a molecular weight of 68 kDa. PRGF also illustrates a faint band around 80 kDa, which is representative of different glycoprotiens, including transferrin, and plasminogen, and a distinct thick band at 14 kDa, indicating the presence of multiple chains of haptoglobin and transthyretin. These results are not surprising, due to the fact that plasma contains large amounts of these proteins. Interestingly, PPP had a similar electrophoretic pattern to that of both FBG and BSA, with the triple banding that is typically seen with FBG and the distinct band at 70 kDa that is characteristic of BSA. Additionally, PPP had additional banding around 80, 25, 18, and other faint bands between 25-50 kDa. These bands most likely represent a multitude of components, including different kinds of glycoproteins (similar to PRGF), various IgG light chains, multiple haptoglobin chains and various lipoproteins (both LDL and HDL). The fact that PPP is made up of mostly albumin, fibrinogen, and immunoglobulins is understandable, due to the fact that these components are the most prevalent proteins in blood.

### 3.2.4 Fluorescent Based Assay.

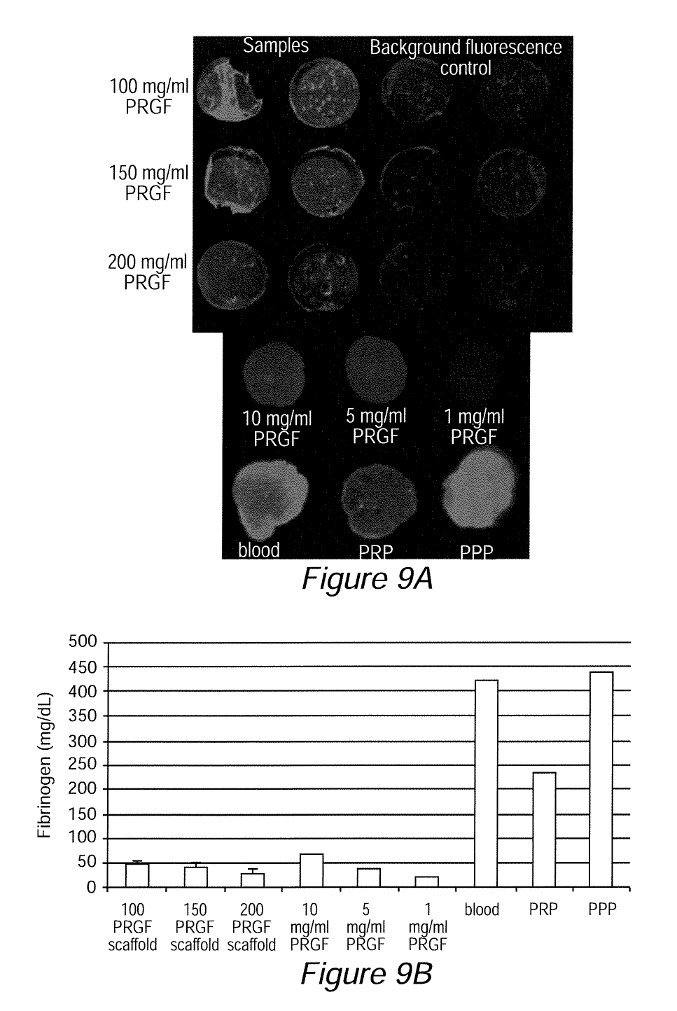

The PVDF membrane that was spotted with 0 to 76 mg/dl of human FBG was scanned by the Odyssey system and fluorescent intensities were acquired (FIG. 9A). A standard curve was obtained from dilutions of human FBG standards for fluorescent assay and showed a linear relationship (R²=0.96, y=1.58x+13.8). Using the standard curve equation, the amount of FBG in each sample was determined. The amount of FBG expressed on scaffolds of pure PRGF ranged from 30-51 mg/dl (FIG. 9B). Specifically, 100 mg/ml PRGF amounted in 51 mg/dl FBG, 150 mg/ml PRGF contained 43 mg/dl FBG, and 200 mg/ml PRGF had 30 mg/dl FBG. PRGF diluted in water at 10, 5 and 1 mg/ml resulted in 67, 37, and 21 mg/dl FBG, respectively. Blood and PPP contained the highest amount of FBG (423 and 440 mg/dl, respectively), while aPRP contained only 234 mg/dl FBG. The values of FBG that were quantified in blood, aPRP, and PPP were expected, and are consistent with previously published data. The presence of FBG and hemoglobin in PRGF may be the reason why this protein is stable enough to form electrospun nanofibers. More specifically, Factor XIII, a stabilizing enzyme of the blood coagulation system that cross-links fibrin, may explain why the electrospun PRGF scaffolds were still intact during the protein release study, even after 35 days in culture. This speculation is based on previous studies, which have demonstrated the ability of FBG and hemoglobin to form electrospun nanofibers from HFP. In addition to the methods presented in this study, the presence of FBG, albumin, and hemoglobin in electrospun PRGF scaffolds was further confirmed by mass spectrometry.

### 3.3 Cell Interaction

Results from DAPI staining of hADSCs cultured on pure PRGF scaffolds reveals cell penetration into the scaffolds after as little as 3 days (FIG. 10). As PRGF electrospinning concentration increases from 100 to 200 mg/ml, it appears there is greater cell migration into the scaffolds, potentially due to the increase in average fiber diameter and subsequent increase in scaffold void space. Regardless, after 10 days it is evident hADSCs have migrated through the entire thickness of the PRGF scaffolds for all concentrations.

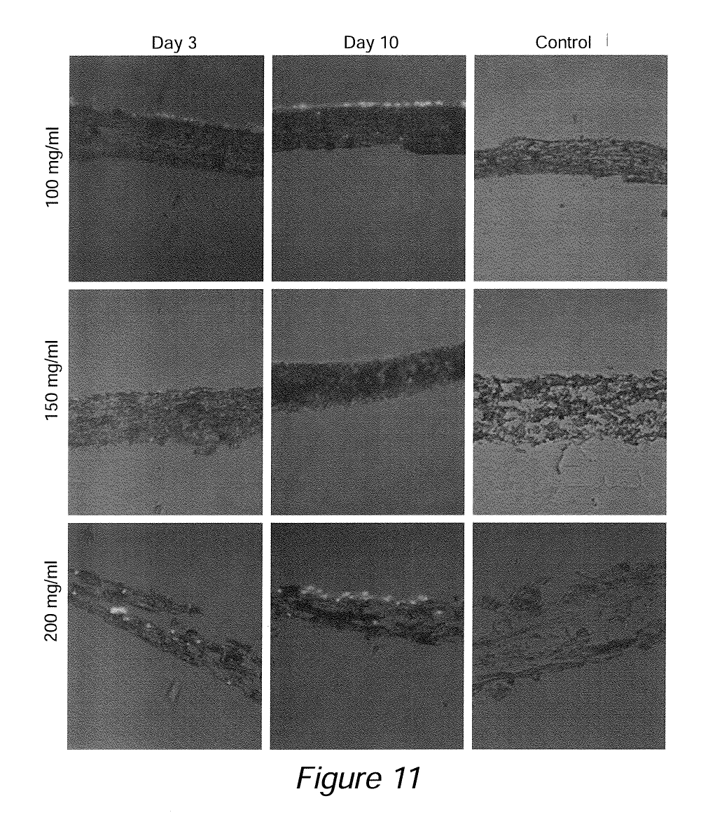

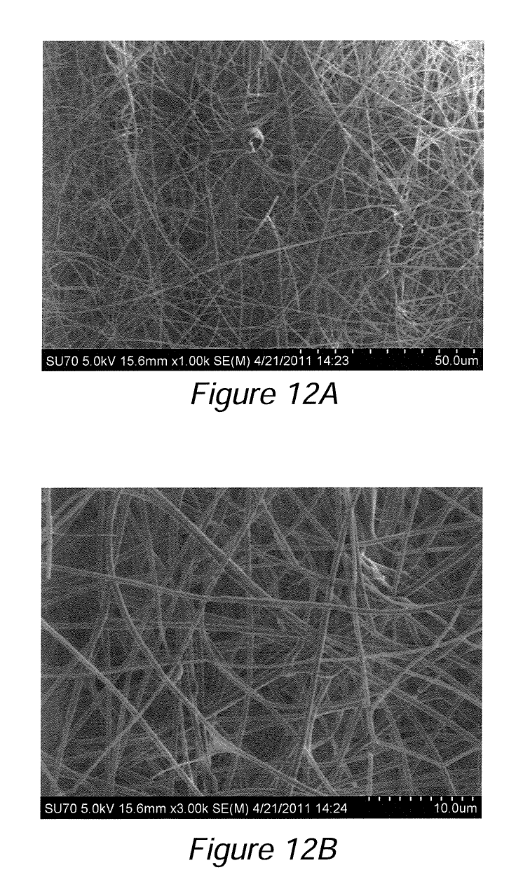

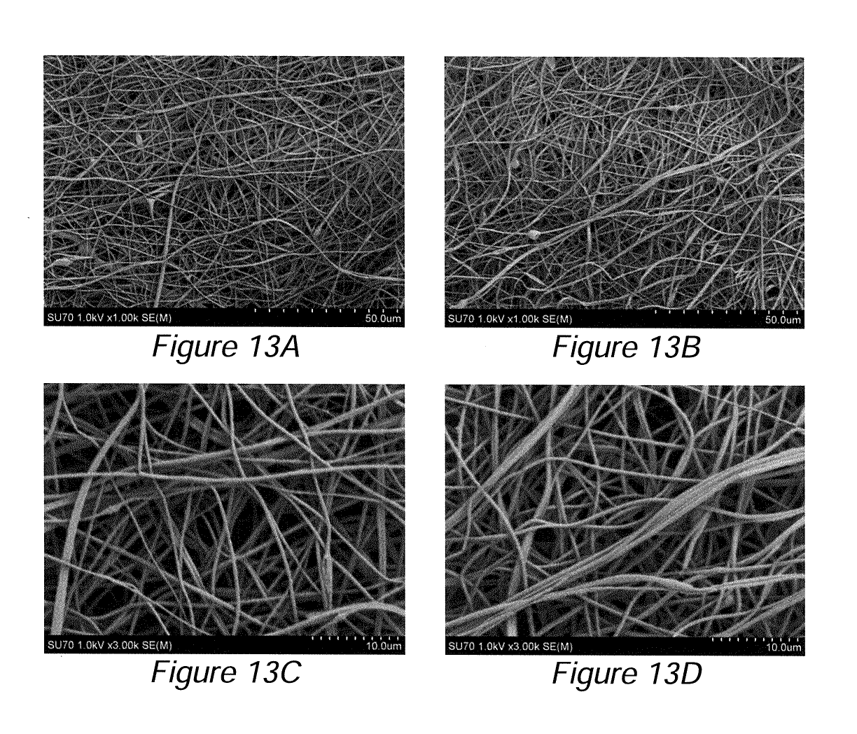

hSMC interaction with PRGF scaffolds is shown in FIG. 11, demonstrating after only 3 days there is cell migration throughout the entire 200 mg/ml PRGF scaffold. 100 mg/ml PRGF scaffolds demonstrated little penetration of hSMCs into the scaffold, with most cells remaining on the surface of the scaffold even after 10 days. Scaffolds of 150 and 200 mg/ml PRGF had complete cellular migration throughout the entire scaffold by day 10. Surprisingly, hSMCs cultured on 150 mg/ml PRGF completely migrated from the surface of the scaffold into the middle region in only 10 days.

The reason for this rapid migration of cells into the scaffold may be two-fold: the presence of an array of chemokines and growth factors found in concentrated amounts in aPRP is most likely chemotactic towards multiple cell types, and the increased void space as PRGF electrospinning concentration increases allows cells to easily migrate into the scaffold. Although not investigated in this manuscript, a previous study conducted by the authors has confirmed the release of RANTES and PDGF-bb from electrospun PRGF fibers (data not shown or published). The release of these growth factors and cytokines, as well as many others contained within PRGF that have been previously shown to be chemotactic and induce cell proliferation, is most likely the reason for enhanced cell infiltration within the scaffolds. Based on the SEMs, it appears the difference in fiber diameters between the PRGF concentrations may affect the porosity of the scaffolds, with increased void space occurring as PRGF concentration increases.

### 4. Conclusion

This example demonstrates, for the first time, the feasibility of creating a nanofibrous scaffold from aPRP. As PRP has gained recent popularity in the clinical setting due to its ability to enhance healing and promote regeneration in an array of tissues clinically, it may prove beneficial when used as a tissue engineering scaffolding material. Not only did electrospun scaffolds of PRGF prove to be stable for extended periods of time in vitro, but they also exhibited a sustained release of proteins known to be important for tissue regeneration for up to 35 days. Additionally, despite the fact that electrospinning is often criticized for its perceived lack of cellular penetration, electrospun PRGF scaffolds promoted rapid cellular infiltration, likely due to the presence of a milieu of growth and chemotactic factors inherent to aPRP. This example shows the tissue engineering potential of electrospun PRGF. In in vivo scenarios, it is expected that multiple regenerative cell types can act concomitantly on the scaffolds in a manner similar to the natural healing cascade through the sustained chemotactic and growth factor gradients eluted.

### Example 3

### Scaffolds with Heparin

Uniaxial Tensile Testing of PCL and PRGF Incorporated Scaffolds with Heparin

### Methods:

Heparin was added to all polymer solutions prior to electrospinning at 0.05% or 0.5% (wt:vol of electrospinning solution). Electrospinning parameters used previously for PCL and PRGF incorporated scaffolds were kept the same. Similar to Example I above, dog-bone shaped samples (overall length of 20 mm, 2.67 mm at its narrowest point, gage length of 7.5 mm, n=5) were punched from each of the electrospun materials. All specimens were placed in a 6 well plate in complete media (DMEM low glucose supplemented with 10% FBS and 1% penicillin/streptomycin) and placed in an incubator (37° C., 5% CO₂) for 1, 7, 14, 21, or 28 days. Samples from each time point were then uniaxially tested to failure at a rate of 10 mm/min using an MTS Bionix 200 testing system with a 100 N load cell (MTS Systems Corp.). Peak stress, modulus, and strain at break were calculated using TestWorks version 4.

### Results:

Results from the mechanical testing showed that all scaffolds (with and without PRGF and/or heparin) maintained their mechanical strength over the 28 day time period. As mentioned above, these results reveal there was little to no degradation occurring in these scaffolds over 28 days. In addition, the incorporation of heparin to the scaffolds had no effect (with the exception of increasing the modulus of PCL and PCL:PRGF scaffolds) on the mechanical properties of the scaffolds as well. Mechanical properties did decrease significantly in scaffolds of PCL:PRGF(100) with and without heparin compared to most other material types at other time points.

### Chemotactic Effect of PRGF and Heparin Incorporated Scaffolds on Macrophages

### Methods:

10 mm diameter discs were punched from electrospun scaffolds incorporated with and without PRGF and/or heparin, disinfected (30 minute soak in ethanol followed by three 10 minute rinses in PBS), and placed in the bottom of a 24-well Transwell plate (8 μm diameter pores, Corning, Inc.). 600 μl of media (RPMI 1640 supplemented with 10% FBS, 1% penicillin/streptomycin) with and without 0.05% or 0.5% heparin was placed in the bottom wells, while the top insert was seeded with 50,000 murine peritoneal macrophages (ATCC, TIB-186) in 200 μl of media for 72 hours (n=3). Following the 72 hour duration, the bottom wells were aspirated and an MTS assay was performed to determine the average cell number in each well.

### Results:

The results of macrophage chemotaxis in response to PRGF and/or heparin incorporated scaffolds showed that the addition of heparin to the media, PCL and PRGF incorporated scaffolds did have an effect on macrophage chemotaxis, hindering their migration over the 72 hour period. With the exception of PCL:PRGF(10), scaffolds and media without heparin induced more migration over those containing heparin, with significantly reduced migration of macrophages occurring for PCL scaffolds (no PRGF) and TCPS.

Chemokine Release from Macrophages Cultured on PRGF and Heparin Incorporated Scaffolds to Determine Phenotype

### Methods:

10 mm diameter discs were punched, disinfected (30 minute soak in ethanol followed by three 10 minute rinses in PBS), and placed in a 48-well plate. Each scaffold was then seeded with 50,000 murine peritoneal macrophages in 500 μl culture media (RPMI 1640 supplemented with 10% FBS, 1% penicillin/streptomycin). In addition, macrophages were also seeded on tissue culture plastic (TCPS) without and with 1 mg/ml PRGF (TCPS:PRGF) and 0.05% and 0.5% heparin. Macrophages were cultured in standard conditions (37° C., 5% CO₂) and supernatant was collected on days 1, 4, 7, and 14. Simultaneously, an MTS assay was performed to determine macrophage number on days 1, 4, 7, and 14. TNF-α (Antigenix America, Inc.) and IL-10 (PeproTech) ELISAs were performed per manufacturer's protocol to quantify the amount M1 (pro-inflammatory, TNF-α) and M2 (pro-regenerative, IL-10) chemokine being released. These results were normalized to amount of chemokine released (ng/ml) per 10,000 cells.

### Results:

IL-10 release from macrophages cultured on PRGF and heparin incorporated scaffolds and TCPS with and without 1 mg/ml PRGF and 0.05% and 0.5% heparin was determined. Overall, the addition of heparin did not appear to affect the release of IL-10 from macrophages cultured on any of the substrates at any time point. Day 1 elicited release from cells cultured on scaffolds of PCL:PRGF(100)+0.05% heparin and PCL:PRGF+0.05% heparin as well as TCPS+0.05% and 0.5% heparin and TCPS:PRGF+0.5% heparin. Release decreased thereafter, and is undetectable by day 14. In contrast to the large amount of IL-10 released from macrophages cultured on PCL:PRGF(10) and PCL:PRGF(100) scaffolds over 14 days, PCL:PRGF(10) and PCL:PRGF(100) scaffolds containing heparin did not elicit a large release of IL-10 from macrophages (with the exception of PCL:PRGF(100)+0.05% heparin). While the addition of heparin to scaffolds in different amounts did not appear have an affect on IL-10 release from macrophages, IL-10 levels were low for almost all substrate types and decline after 1 day, suggesting a change in the macrophage phenotype.

TNF-α release from macrophages cultured on PRGF incorporated scaffolds with heparin was investigated. With the exception of PCL:PRGF(100)+heparin scaffolds, release of TNF-α from macrophages cultured on scaffolds was low at days 1-7, and increased by day 14. Release from cells cultured on TCPS and TCPS:PRGF with heparin appeared constant over the 14 days. Similar to IL-10, the addition of heparin in different amounts did not appear to have a significant affect on TNF-α release between substrates, except PCL:PRGF+0.5% heparin at day 14. Due to the fact that IL-10 release decreased throughout the 14 days and TNF-α release increased throughout 14 days, heparin may be driving macrophages from the M2 phenotype towards the M1 phenotype.

### Example 4

### Electrospinning with Silk, PRGF, and Polyethylene Oxide (PEO)

Electrospinning Silk: PRGF from Water (80 mg/mL:250 mg/mL)

### Methods:

250 mg/mL PRGF was added to an 80 mg/mL aqueous silk solution. 50 mg/mL PEO (900,000 MW) (Sigma Adrich) aqueous solution was then added to the aqueous solution to make a 40:40:20 silk:PRGF:PEO weight:volume solution. The solution remained on a shaker plate at room temperature until fabrication to ensure thorough blending. Prior to electrospinning, 0.5 mL PEO in ethanol was electrospun onto a rectangular metal mandrel (2.5 cm wide×10.2 cm long×0.3 cm thick) to help the aqueous solution electrospin properly. The aqueous solution was loaded into a 3 mL Becton Dickinson syringe with a blunt-end 18 gauge needle and dispensed at a rate of 1 mL/hr by a KD Scientific syringe pump. A charging voltage of 30 kV was applied to the needle and a −10 kV charging voltage was applied to the 15.2 cm diameter aluminum target behind the mandrel. The mandrel rotated at 400 rpm covering a distance of 6 cm/s with an air gap distance of 8 in between the needle and mandrel. All electrospinning was conducted at room temperature. The samples were immediately cut off the mandrel using a razor blade and stored in a desiccator chamber.

Electrospinning Silk:PRGF from Water (80 mg/mL:300 Mg/mL)

### Methods:

300 mg/mL PRGF was added to an 80 mg/mL aqueous silk solution. 75 mg/mL PEO (900,000 MW) (Sigma Adrich) aqueous solution was then added to the aqueous solution to make an 40:40:20 silk:PRGF:PEO weight:volume solution. The solution remained on a shaker plate at room temperature until fabrication to ensure thorough blending. Prior to electrospinning, 0.5 mL PEO in ethanol was electrospun onto a rectangular metal mandrel (2.5 cm wide×10.2 cm long×0.3 cm thick) to help the aqueous solution electrospin properly. The solution was loaded into a 3 mL Becton Dickinson syringe with a blunt-end 18 gauge needle and dispensed at a rate of 1 mL/hr by a KD Scientific syringe pump. A charging voltage of 30 kV was applied to the needle and a −10 kV charging voltage was applied to the 15.2 cm diameter aluminum target behind the mandrel. The mandrel rotated at 400 rpm covering a distance of 6 cm/s with an air gap distance of 8 in between the needle and mandrel. All electrospinning was conducted at room temperature. The samples were immediately cut off the mandrel using a razor blade and stored in a desiccator chamber.

### Results:

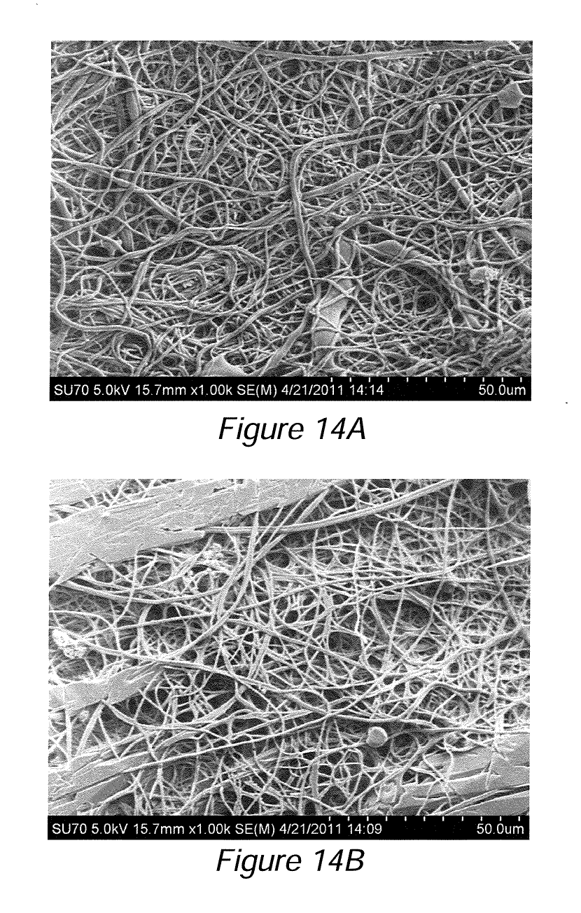

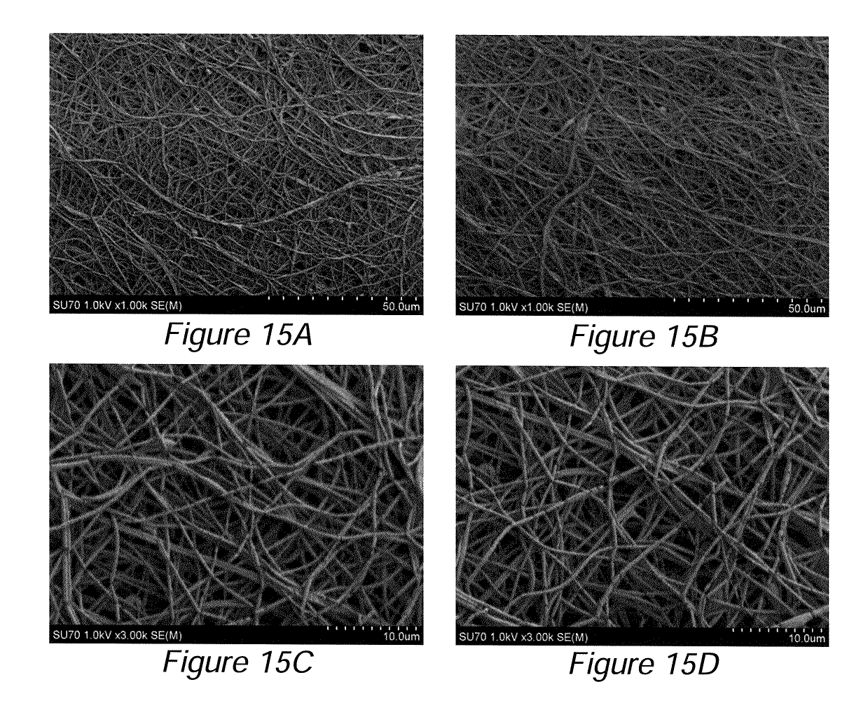

Electrospinning 80 mg/mL:250 mg/mL aqueous silk:PRGF solution with 20% PEO resulted in randomly oriented, non-woven scaffolds (FIG. 13A-D). Slightly varying the parameters (80 mg/mL: 300 mg/mL aqueous silk:PRGF) still resulted in fibrous, randomly-oriented scaffolds as seen in FIGS. 14A and B.

Electrospinning PRGF from an aqueous solution also produced a fibrous scaffold (FIG. 15A-D). 300 mg/mL PRGF was added to deionized water to make an aqueous electrospinning solution. 75 mg/mL PEO (900,000 MW) (Sigma Adrich) aqueous solution was then added to the aqueous PRGF solution to make a 60:40 PRGF:PEO weight:volume solution. The solution remained on a shaker plate at room temperature until fabrication to ensure thorough blending. Prior to electrospinning, 0.5 mL PEO in ethanol was electrospun onto a rectangular metal mandrel (2.5 cm wide×10.2 cm long×0.3 cm thick) to help the aqueous solution electrospin properly. The electrospinning solution was loaded into a 3 mL Becton Dickinson syringe with a blunt-end 18 gauge needle and dispensed at a rate of 0.75 mL/hr by a KD Scientific syringe pump. A charging voltage of 30 kV was applied to the needle and a −10 kV charging voltage was applied to the 15.2 cm diameter aluminum target behind the mandrel.

This example demonstrates that PRGF can be electrospun from an aqueous solution, using at least one carrier molecule such as SF or PEO.

### Example 5

### Uniaxial Tensile Testing of Crosslinked Fibers

### Methods:

Fibers were electrospun as described in Example 4. Dog bone-shaped samples (2.75 mm wide at the most narrow space and 7.5 mm long) were punched out of the electrospun samples. The dog bones were crosslinked with either EDC (50 mM, room temperature) or genipin (30 mM, 37° C.) for 24 hours and then PBS for another hour prior to testing. The samples were tested to failure on a MTS Bionix 200 testing system (MTS Systems Corp) at an extension rate of 10.0 mm/min. The elastic modulus strain at break and peak stress were calculated by the MTS software TestWorks 4.0 and recorded.

### Results:

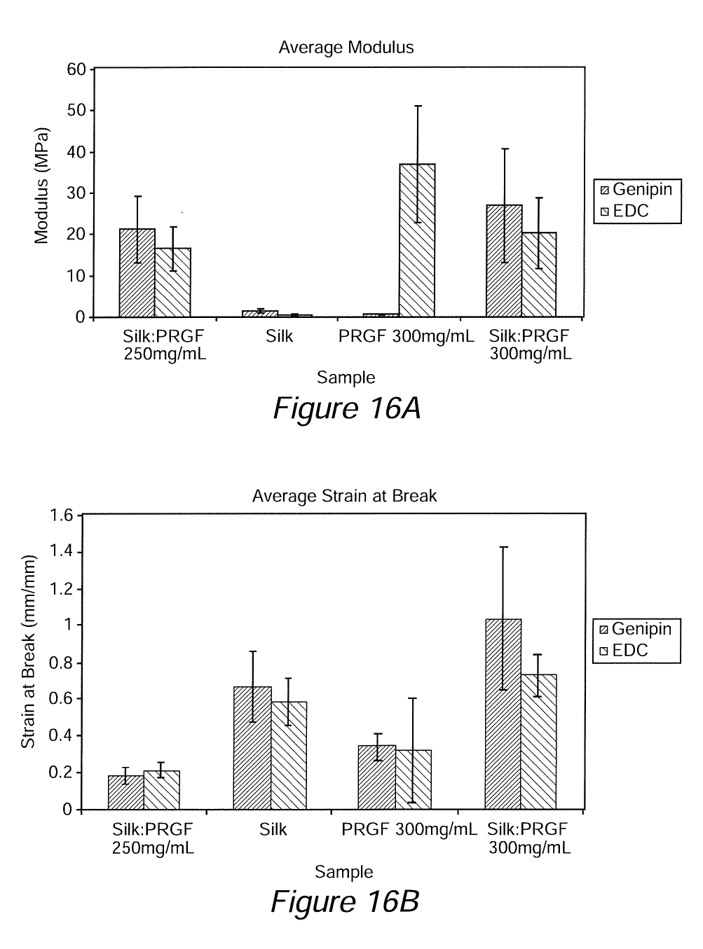

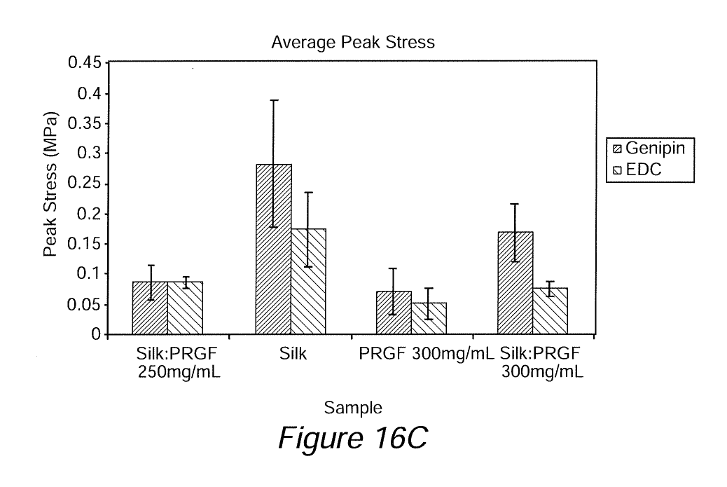

Uniaxial tensile testing compared the crosslinking effects of EDC and genipin. The effect of PRGF concentration when blended with silk was also observed in the average modulus, strain at break and peak stress (FIG. 16A-C). These results demonstrate that the inclusion of PRGF, cross-linked with either EDC or genipin, increases material modulus over SF. Strain at break is also improved over pure SF when PRGF is electrospun at 300 mg/ml. Most importantly these results demonstrate that electrospun PRGF can be cross-linked using cross-linking agents commonly used in tissue engineering applications. This may be indicative of the array of proteins present in the PRGF, due to the fact that genipin and EDC are only effective at cross-linking specific proteins (collagen, fibrinogen, elastin, etc.).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. An electrospun fiber comprising either or both of platelet-rich plasma (PRP) and activated platelet-rich plasma (aPRP).

2. The electrospun fiber of claim 1, wherein said electrospun fiber further comprises at least one synthetic polymer.

3. The electrospun fiber of claim 2, wherein said at least one synthetic polymer is selected from the group consisting of poly(glycolic acid) (PGA), polycaprolactone (PCL), poly(lactic acid) (PLA), poly(lactic-co-glycolic acid) (PLGA), polydioxanone (PDO) and polyethylene oxide (PEO).

4. The electrospun fiber of claim 1, wherein said electrospun fiber further comprises at least one natural polymer.

5. The electrospun fiber of claim 4, wherein said at least one natural polymer is silk fibroin.

6. The electrospun fiber of claim 1, wherein said electrospun fiber is formed from aPRP.

7. A method for the sustained delivery of one or more components of PRP to a patient in need thereof, comprising the step of
providing to said patient a construct comprising electrospun aPRP fibers.

8. The method of claim 7, wherein said one or more components of PRP include a substance selected from the group consisting of platelet-derived growth factor (PDGF), transforming growth factor-β (TGF-β), and RANTES (Regulated upon Activation, Normal T-cell Expressed, and Secreted) factors.

9. The method of claim 7, wherein said providing step includes applying said construct to a site of injury.

10. The method of claim 7, wherein said PRP is autologous PRP.

11. The method of claim 7, wherein said PRP is allogenic PRP.

12. The method of claim 7, wherein said providing step includes electrospinning said construct into a site of injury.

13. A method of making electrospun aPRP fibers, comprising the steps of
obtaining PRP;
activating said PRP, thereby forming activated PRP (aPRP);
dehydrating said aPRP, thereby forming dehydrated aPRP;
dissolving said dehydrated aPRP in a solvent suitable for electrospinning, thereby forming an aPRP electrospinning solution, and
electrospinning said aPRP electrospinning solution into electrospun aPRP fibers.

14. The method of claim 13, wherein said step of activating is carried out by subjecting said PRP to at least one cycle of freezing, thawing, and freezing.

15. The method of claim 13, wherein said step of dehydration is carried out by freeze-drying said aPRP.

16. A method of delivering activated platelet-rich plasma (aPRP) to a patient in need thereof, comprising the step of
electrospinning said aPRP to form electrospun aPRP fibers; and
providing said electrospun aPRP fibers to said patient.

17. The method of claim 16, wherein said aPRP fibers provide sustained delivery of said aPRP to said patient.

18. A method of forming engineered tissue, comprising the steps of
exposing tissue-forming cells to a tissue-engineering scaffold comprising electrospun aPRP fibers under conditions that allow said cells to migrate on, infiltrate and/or proliferate on or within said tissue-engineering scaffold, thereby forming said engineered tissue.

19. The method of claim 18, wherein said tissue-forming cells are in vivo.

20. The method of claim 18, wherein said tissue-forming cells are in vitro.

21. A tissue engineering scaffold formed from electrospun fibers comprising activated platelet-rich plasma (aPRP).

22. Electrospun activated platelet-rich plasma (aPRP).
